(19) United States
(12) Patent Application Publication
(10) Pub. No.: US 2026/0211501 A1
(43) Pub. Date: Jul. 23, 2026

(54) AUTOMATICALLY ALIGNING BAND PORTIONS OF A WRIST-WEARABLE DEVICE

(71) Applicant: Meta Platforms Technologies, LLC, Menlo Park, CA (US)

(72) Inventors: Michael Garcia, Redondo Beach, CA (US); Geremy Patterson, La Jolla, CA (US); Yu-Nan Su, Sunnyvale, CA (US); Andrew Rosenkranz, Westfield, NJ (US); Thompson Nguyen, Jersey City, NJ (US); Shubham Aggarwal, New York, NY (US); Claudia Moreira, Fairfield, NJ (US); John Bernard Ardisana, II, Farmington Hills, MI (US)

(21) Appl. No.: 19/449,390

(22) Filed: Jan. 14, 2026

Related U.S. Application Data
(60) Provisional application No. 63/746,360, filed on Jan. 17, 2025.

(51) Int. Cl. G06F 3/01; A61B 5/00; A61B 5/296

(52) U.S. Cl. CPC G06F 3/015; G06F 3/014; G06F 3/017; A61B 5/296; A61B 5/681

(57) ABSTRACT

A wrist-wearable device including a band for sensing neuromuscular signals. The device comprises a first band section and a second band section. A plurality of neuromuscular-signal sensors are disposed within the first band section and configured to contact a wrist of a user on a first side of the first band section. A first attachment magnet is disposed within the first band section, having a first central magnetic portion with a first magnetic polarity disposed between first lateral magnetic portions each having a second magnetic polarity. A plurality of second attachment magnets are disposed within the second band section, each having a second central magnetic portion with the second magnetic polarity disposed between second lateral magnetic portions each having the first magnetic polarity. Magnetic attractive forces between the central and lateral magnetic portions magnetically couple the second band section with the first band section.

### RELATED APPLICATION

This application claims priority to U.S. Provisional Application No. 63/746,360, titled “Automatically Aligning Band Portions Of A Wrist-Wearable Device,” filed Jan. 17, 2025, which are hereby incorporated by reference in its entirety.

### TECHNICAL FIELD

This relates generally to self-aligning band portions of a wrist-wearable device, where the self-alignment ensures sufficient skin contact and pressure for sensors of the band portion when donned on a wrist of a wearer.

### BACKGROUND

The evolution of wrist-wearable device band portion(s) includes making the band a more functional part of the wrist-wearable device. The evolution includes adding features to the band such as the integration of electronics that can detect one or more signals and transmit them to the wrist-wearable device. However, as additional electronics are integrated into the band, the integration remains a challenge, particularly in maintaining functionality and comfort. Conventional wrist-wearable device bands typically rely on traditional watchband mechanisms to secure the band around the wrist. These traditional mechanisms fail to (i) comfortably provide a wide range of sizes for users and (ii) provide consistent alignment of the band portions such that the electronic components do not suffer from interference (e.g., magnets and sensors interfering).

As such, there is a need to address one or more of the above-identified challenges. A brief summary of solutions to the issues noted above is described below.

### SUMMARY

To address the above-described challenges, an example wrist-wearable device described herein can include at least two band sections with magnetic-attachment structures that have opposing polarity portions. When coupled via magnetic attraction, the opposing polarity portions of the at least two magnetic-attachment structures self-align the at least two band sections such that they are centered, and sensors in at least one band section contact the skin of a user for accurate signal detection and improved comfort.

In yet another example that resolves the challenges described above, a wrist-wearable device can include a band for sensing neuromuscular signals. The wrist-wearable device includes a first band section including neuromuscular-signal sensors configured to be in contact with a wrist of a user on a first side of the first band section. The first band section of the wrist-wearable device further includes a second side, opposite the first side of the first band section, which includes a first magnetic-attachment structure having a first magnetic portion with a first magnetic polarity in between a second magnetic portion having a second magnetic polarity. The wrist-wearable device further includes a second band section that includes a plurality of second magnetic-attachment structures each having a third magnetic portion with the second magnetic polarity placed in between a fourth magnetic portion with the first magnetic polarity. The second band section is configured to magnetically couple with the first band section by way of a magnetic attractive force between the first magnetic portion of the first band section and respective third magnetic portions of the plurality of second magnetic-attachment structures, and a magnetic attractive force between the second magnetic portion of the first band section and respective fourth magnetic portions of the plurality of second magnetic-attachment structures.

The features and advantages described in the specification are not necessarily all-inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIG. 1 illustrates an example embodiment of two band sections magnetically coupled to each other, in accordance with some embodiments.

FIG. 2 illustrates an example method of manufacturing of a band section of a wrist-wearable device, in accordance with some embodiments.

FIG. 3 illustrates an example embodiment integrating electronics into the band portion of a wrist-wearable device, in accordance with some embodiments.

FIG. 4 illustrates an example method of coupling a neuromuscular-signal sensor to a band portion of a wrist-wearable device, in accordance with some embodiments.

FIG. 5 illustrates an example embodiment of a method to secure the band of the wrist-wearable device, in accordance with some embodiments.

FIG. 6 shows an example method flow chart for coupling the second band section to the first band section of the wrist-wearable device via magnetic properties, in accordance with some embodiments.

FIGS. 7A, 7B, 7C-1, and 7C-2 illustrate example MR and AR systems, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, similar reference numerals may be used to denote similar features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

### Overview

Embodiments of this disclosure can include or be implemented in conjunction with various types of extended realities (XRs) such as mixed-reality (MR) and augmented-reality (AR) systems. MRs and ARs, as described herein, are any superimposed functionality and/or sensory-detectable presentation provided by MR and AR systems within a user's physical surroundings. Such MRs can include and/or represent virtual realities (VRs) and VRs in which at least some aspects of the surrounding environment are reconstructed within the virtual environment (e.g., displaying virtual reconstructions of physical objects in a physical environment to avoid the user's colliding with the physical objects in a surrounding physical environment). In the case of MRs, the surrounding environment that is presented through a display is captured via one or more sensors configured to capture the surrounding environment (e.g., a camera sensor, time-of-flight (ToF) sensor). While a wearer of an MR headset can see the surrounding environment in full detail, they are seeing a reconstruction of the environment reproduced using data from the one or more sensors (i.e., the physical objects are not directly viewed by the user). An MR headset can also forgo displaying reconstructions of objects in the physical environment, thereby providing a user with an entirely VR experience. An AR system, on the other hand, provides an experience in which information is provided, e.g., through the use of a waveguide, in conjunction with the direct viewing of at least some of the surrounding environment through a transparent or semi-transparent waveguide(s) and/or lens(es) of the AR glasses. Throughout this application, the term “extended reality (XR)” is used as a catchall term to cover both ARs and MRs. In addition, this application also uses, at times, a head-wearable device or headset device as a catchall term that covers XR headsets such as AR glasses and MR headsets.

As alluded to above, an MR environment, as described herein, can include, but is not limited to, non-immersive, semi-immersive, and fully immersive VR environments. As also alluded to above, AR environments can include marker-based AR environments, markerless AR environments, location-based AR environments, and projection-based AR environments. The above descriptions are not exhaustive and any other environment that allows for intentional environmental lighting to pass through to the user would fall within the scope of an AR, and any other environment that does not allow for intentional environmental lighting to pass through to the user would fall within the scope of an MR.

The AR and MR content can include video, audio, haptic events, sensory events, or some combination thereof, any of which can be presented in a single channel or in multiple channels (such as stereo video that produces a three-dimensional effect to a viewer). Additionally, AR and MR can also be associated with applications, products, accessories, services, or some combination thereof, which are used, for example, to create content in an AR or MR environment and/or are otherwise used in (e.g., to perform activities in) AR and MR environments.

Interacting with these AR and MR environments described herein can occur using multiple different modalities, and the resulting outputs can also occur across multiple different modalities. In one example AR or MR system, a user can perform a swiping in-air hand gesture to cause a song to be skipped by a song-providing application programming interface (API) providing playback at, for example, a home speaker.

A hand gesture, as described herein, can include an in-air gesture, a surface-contact gesture, and/or other gestures that can be detected and determined based on movements of a single hand (e.g., a one-handed gesture performed with a user's hand that is detected by one or more sensors of a wearable device (e.g., electromyography (EMG) and/or inertial measurement units (IMUs) of a wrist-wearable device, and/or one or more sensors included in a smart textile wearable device) and/or detected via image data captured by an imaging device of a wearable device (e.g., a camera of a head-wearable device, an external tracking camera setup in the surrounding environment)). “In-air” generally includes gestures in which the user's hand does not contact a surface, object, or portion of an electronic device (e.g., a head-wearable device or other communicatively coupled device, such as the wrist-wearable device); in other words, the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single-or double-finger tap on a table, on a user's hand or another finger, on the user's leg, a couch, a steering wheel). The different hand gestures disclosed herein can be detected using image data and/or sensor data (e.g., neuromuscular signals sensed by one or more biopotential sensors (e.g., EMG sensors) or other types of data from other sensors, such as proximity sensors, ToF sensors, sensors of an IMU, capacitive sensors, strain sensors) detected by a wearable device worn by the user and/or other electronic devices in the user's possession (e.g., smartphones, laptops, imaging devices, intermediary devices, and/or other devices described herein).

The input modalities as alluded to above can be varied and are dependent on a user's experience. For example, in an interaction in which a wrist-wearable device is used, a user can provide inputs using in-air or surface-contact gestures that are detected using neuromuscular-signal sensors of the wrist-wearable device. In the event that a wrist-wearable device is not used, alternative and entirely interchangeable input modalities can be used instead, such as camera(s) located on the headset/glasses or elsewhere to detect in-air or surface-contact gestures or inputs at an intermediary processing device (e.g., through physical input components (e.g., buttons and trackpads)). These different input modalities can be interchanged based on both desired user experiences, portability, and/or a feature set of the product (e.g., a low-cost product may not include hand-tracking cameras).

While the inputs are varied, the resulting outputs stemming from the inputs are also varied. For example, an in-air gesture input detected by a camera of a head-wearable device can cause an output to occur at a head-wearable device or control another electronic device different from the head-wearable device. In another example, an input detected using data from a neuromuscular-signal sensor can also cause an output to occur at a head-wearable device or control another electronic device different from the head-wearable device. While only a couple of examples are described above, one skilled in the art would understand that different input modalities are interchangeable along with different output modalities in response to the inputs.

Specific operations described above may occur as a result of specific hardware. The devices described are not limiting, and features on these devices can be removed or additional features can be added. The different devices can include one or more analogous hardware components. For brevity, analogous devices and components are described herein. Any differences in the devices and components are described below in their respective sections.

As described herein, a processor (e.g., a central processing unit (CPU) or microcontroller unit (MCU)) is an electronic component that is responsible for executing instructions and controlling the operation of an electronic device (e.g., a wrist-wearable device, a head-wearable device, a handheld intermediary processing device (HIPD), a smart textile-based garment, or other computer system). There are several types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., VR animations, such as three-dimensional modeling); (iv) a field-programmable gate array (FPGA) that can be programmed and reconfigured after manufacturing and/or customized to perform specific tasks, such as signal processing, cryptography, and machine learning; or (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronic components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IOT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs. As described herein, a graphics module is a component or software module that is designed to handle graphical operations and/or processes and can include a hardware module and/or a software module.

As described herein, memory refers to electronic components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid-state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, and other non-volatile solid-state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include (i) profile data, including user account data, user settings, and/or other user data stored by the user; (ii) sensor data detected and/or otherwise obtained by one or more sensors; (iii) media content data, including stored image data, audio data, documents, and the like; (iv) application data, which can include data collected and/or otherwise obtained and stored during use of an application; and/or (v) any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronic components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) pogo pins, which may be small, spring-loaded pins configured to provide a charging interface; (v) wireless charging interfaces; (vi) global-positioning system (GPS) interfaces; (vii) Wi-Fi interfaces for providing a connection between a device and a wireless network; and (viii) sensor interfaces.

As described herein, sensors are electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device, such as a simultaneous localization and mapping (SLAM) camera); (ii) biopotential-signal sensors; (iii) IMUs for detecting, for example, angular rate, force, magnetic field, and/or changes in acceleration; (iv) heart rate sensors for measuring a user's heart rate; (v) peripheral oxygen saturation (SpO₂) sensors for measuring blood oxygen saturation and/or other biometric data of a user; (vi) capacitive sensors for detecting changes in potential at a portion of a user's body (e.g., a sensor-skin interface) and/or the proximity of other devices or objects; (vii) sensors for detecting some inputs (e.g., capacitive and force sensors); and (viii) light sensors (e.g., ToF sensors, infrared light sensors, or visible light sensors), and/or sensors for sensing data from the user or the user's environment. As described herein, biopotential-signal-sensing components are devices used to measure electrical activity within the body (e.g., biopotential-signal sensors). Some types of biopotential-signal sensors include (i) electroencephalography (EEG) sensors configured to measure electrical activity in the brain to diagnose neurological disorders; (ii) electrocardiogram EKG) sensors configured to measure electrical activity of the heart to diagnose heart problems; (iii) EMG sensors configured to measure the electrical activity of muscles and diagnose neuromuscular disorders; and (iv) electrooculography (EOG) sensors configured to measure the electrical activity of eye muscles to detect eye movement and diagnose eye disorders.

As described herein, an application stored in the memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications; (x) camera applications; (xi) web-based applications; (xii) health applications; (xiii) AR and MR applications; and/or (xiv) any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). A communication interface can refer to a software layer that enables different software programs to communicate with each other (e.g., APIs and protocols such as HTTP and TCP/IP).

As described herein, a graphics module is a component or software module that is designed to handle graphical operations and/or processes and can include a hardware module and/or a software module.

As described herein, non-transitory computer-readable storage media are physical devices or storage media that can be used to store electronic data in a non-transitory form (e.g., such that the data is stored permanently until it is intentionally deleted and/or modified).

### Magnetically Coupling the Band Sections of a Wrist-wearable Device

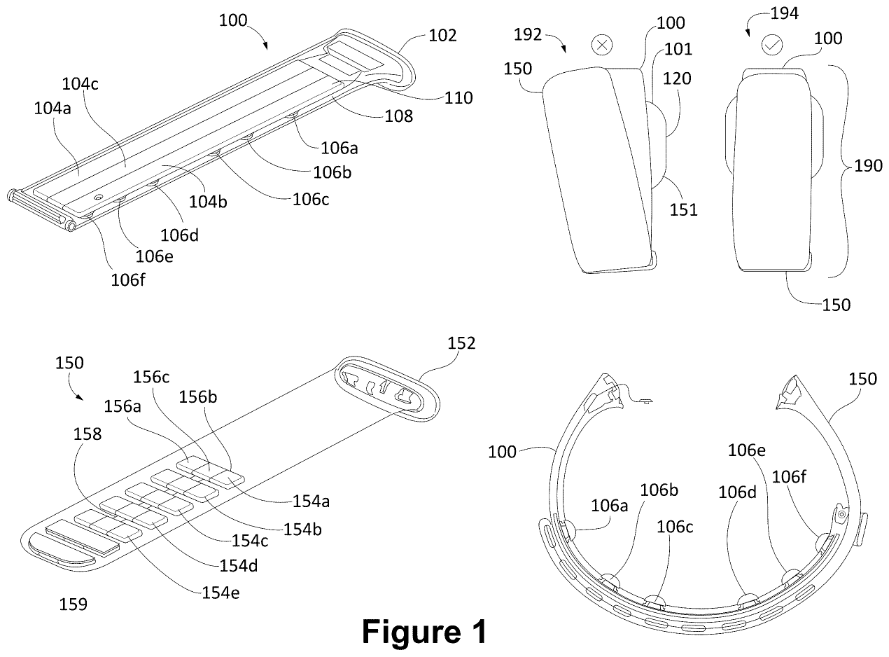

FIG. 1 illustrates an example embodiment of at least two band sections of a wrist-wearable device that can be magnetically coupled to each other, in accordance with some embodiments. FIG. 1 further illustrates a first band section 100 and a second band section 150 that are coupled to a computing system (also referred to as a “capsule 120”) of a wrist-wearable device (e.g., a smartwatch) via a first connection point 102 and a second connection point 152. In some embodiments, the first band section 100 is connected to a first side of a capsule 101 via the first connection point 102, and the second band section 150 is connected to a second side of the capsule 151, opposite the first side of the capsule 101, via the second connection point 152. In some embodiments, the first band section 100 and the second band section 150 are configured to couple to each other via magnetic properties (e.g., magnetic properties from ferromagnetic materials or paramagnetic materials) such that the wrist-wearable device is secured on the wrist of a user. In some embodiments, the magnetic properties are provided by magnetic alloy-based materials, magnetic composite materials (e.g., rubber-based, ceramic-based, etc.) that include magnetic materials (e.g., ferromagnetic or paramagnetic materials), or any combination thereof). For example, an example composite material can be part the first band section 100 and the second band section 150, which can be made of liquid silicon rubber mixed with any amount of magnetic particles (in a predetermined ratio and a predetermined size) to create homogenous blend of magnetic rubber material. In some embodiments, the inclusion of the tiny magnetic particles does not substantially alter the material properties of the liquid silicon rubber. In the following descriptions “magnets” are used as a catchall term, and one skilled in the art will understand that the term magnet can refer to any of the materials with magnetic properties listed above, including composite materials, alloys, or individual materials.

In some embodiments, the first band section 100 includes one or more neuromuscular-signal sensors 106a-106f on a first side of the first band section 108 configured to couple to a portion of the user's body (e.g., skin on the user's wrist). A second side of the first band section 110, opposite the first side of the first band section 108, includes a first magnetic structure including a first magnetic portion 104a, a second magnetic portion 104b, and a third magnetic portion 104c. The first magnetic structure extends along a longitudinal portion of the first band section 100 such that the first magnetic structure covers a predetermined circumference of a wrist of the user (e.g., a major axis of the first band section 100). As described in detail below, when the second band portion 150 is magnetically coupled to the first band portion 100, the band circumference can be adjusted for different size wrists by adjusting the placement of the first band portion 100 relative to the second band portion 150 (e.g., a different alignment of magnets).

In some embodiments, the first magnetic structure includes three magnetic portions 104a, 104b, and 104c arranged with alternating polarities as described above. However, it should be understood that the number of adjacent alternating poles is not limited to three. In alternative embodiments, the first magnetic structure may include four, five, or more magnetic portions arranged with alternating polarities. Similarly, each magnetic-attachment structure of the second band section 150 may include corresponding additional magnetic portions with alternating polarities to complement the first magnetic structure. The alternating polarity arrangement, regardless of the number of magnetic portions, facilitates self-alignment between the first band section 100 and the second band section 150 when the band sections are brought into proximity with one another.

The first magnetic portion 104a and the second magnetic portion 104b (e.g., outer magnets) are a first magnetic polarity and the third magnetic portion 104c (e.g., inner magnet) is a second magnetic polarity opposite the first polarity. The third magnetic portion 104c is disposed in between the first magnetic portion 104a and the second magnetic portion 104b. In some embodiments, each respective magnetic portion 104 a-104c ranges between 3 mm and 10 mm wide, e.g., each being 6 mm wide. In some embodiments, the magnetic portions can each have varied sizes (e.g., magnetic portion 104a and magnetic portion 104b being wider than magnetic portion 104c).

The second band section 150 includes a second arrangement of magnets that includes one or more magnetic-attachment structure(s). In one example, the second arrangement of magnets includes magnetic-attachment structures 154a-154e that are spaced a predetermined distance away from each other. These magnetic-attachment structures 154a-154e are configured to interface with the magnet portions 104a-104c to thereby allow for the second band section 150 to be coupled to the first band section 100. The predetermined spacing between the magnetic-attachment structures 154a-154e is further configured to provide additional comfort to the user while wearing the wrist-wearable device. For example, the spacing between the magnetic structures 154a-154e is done evenly such that one or more neuromuscular-signal sensors 106a-106f apply an even amount of force on the skin of the user when the wrist-wearable device is worn. In a non-limiting example, FIG. 1 illustrates five magnetic-attachment structures 154a-154e linked by a connection layer 158. In some embodiments, the connection layer 158 is a strain relief layer formed of one or more materials (e.g., a layer composed of a manufactured fiber that is spun from a liquid crystal polymer (LCP)). In some embodiments, the one or more magnetic-attachment structures 154a-154e are hidden in the second band section 150 and not visible to the user wearing the wrist-wearable device. This is achieved through a uniform thickness of the material surrounding the two or more magnetic-attachment structures 154a-154e.

Each one of magnetic-attachment structures 154a-154e includes a first magnetic portion 156a, a second magnetic portion 156b, and a third magnetic portion 156c. The first magnetic portion 156a and the second magnetic portion 156b are a second magnetic polarity and the third magnetic portion 156c is a first magnetic polarity. The third magnetic portion 156c is disposed in between the first magnetic portion 156a and the second magnetic portion 156b. In some embodiments, the first magnetic portion 156a and the second magnetic portion 156b are the same polarity as the third magnetic portion 104c of the first magnetic structure, and the third magnetic portion 156c is the same polarity as the first magnetic portion 104a and the second magnetic portion 104b of the first magnetic structure. Similarly, the first magnetic portion 156a and the second magnetic portion 156b are an opposite polarity to the first magnetic portion 104a and the second magnetic portion 104b of the first magnetic structure, and the third magnetic portion 156c is an opposite polarity to the third magnetic portion 104c. More specifically, the first magnetic structure included in the first band section 100 and each respective magnetic-attachment structure 154a-154e of the second magnetic structure included in the second band section 150 are configured to have opposite magnetic polarities such that the first band section 100 and the second band section 150 can couple via a magnetic attractive force.

In some embodiments, the respective magnetic portions 104a-104b and the magnetic-attachment structures 154a-154e are made using a liquid silicone rubber mixed with magnetic particles in a desired ratio to create a homogenous blend of magnetic rubber material. The material is then compression molded into a specific shape (e.g., a shape matching the first band portion 100 and the second band portion 150) and permanently magnetized using a uni-directional field. The uni-directional field ensures that the magnetic force is stronger in the direction of the tail retention (e.g., ends opposite the first connection point 102 and/or the second connection point 152). As described above with respect to the respective magnetic portions 104a-104b and the magnetic-attachment structures 154a-154e, respective 3-pole designs of the first band portion 100 and the second band portion 150 have opposite polarities such that there are magnetic attractive forces between the first band portion 100 and the second band portion 150 that self-align the bands and comfortably couple the first and second band portions 100 and 150 to the skin of the user.

When a user puts on the wrist-wearable device 190 and the first band section 100 and the second band section 150 are aligned, they remain coupled due to magnetic coupling (e.g., via a magnetic attractive force). The excess band (e.g., tail portion 159) in the second band section 150 automatically lies down along the first band portion 100 with minimal effort from the user. In some embodiments, the tail portion 159 of the second band portion 150 also includes magnetic properties. The tail portion 159 can include one or more magnetized segments. In some embodiments, the tail portion 159 includes stronger magnetic properties such that when the tail portion 159 is coupled to the first band portion 100, it requires additional force to remove the tail portion 159 from the first band portion 100. Using this method allows for high retention force at the tail and ensures that excess tail stays retained during active motion (e.g., the end opposite the second connection point 152 lies substantially flat along the end opposite the first connection point 102).

In some embodiments, when the first band section 100 and the second band section 150 overlap, they self-align because of the polarity of the respective magnetic structures contained in the first and second band portions 100 and 150. FIG. 1 further illustrates the wrist-wearable device 190 at a first point in time 192 where the first and second band portions 100 and 150 are misaligned and at a second point in time 194 where the first and second band portions 100 and 150 are aligned. At the first point in time 192 where the first and second band portions 100 and 150 are overlapping but are not aligned, the polarity of the magnetic structures contained in the first and second band portions 100 and 150 automatically re-align the first and second band portions 100 and 150, which is shown at the second point in time 194. For example, if the first and second band portions 100 and 150 are overlapped such that the third magnetic portion 104c of the first band section 100 and the first magnetic portion 156a or the second magnetic portion 156b of magnetic-attachment structure 154e are positioned such that they are adjacent, there is a magnetic repulsive force therebetween as they each have the same magnetic polarity. Therefore, the first and second band sections 100 and 150 cannot magnetically couple in this configuration. In this example, the repulsive force created between the magnetic sections that contain the same magnetic polarity automatically move and align the first and second band sections 100 and 150 such that the third magnetic portion 104c of the first band section 100 is magnetically coupled to the third magnetic portion 156c of the second band section 150, the first magnetic portion 104a is magnetically coupled to a respective second magnetic portion 156b, and the second magnetic portion 104b is magnetically coupled to a respective first magnetic portion 156a. The successful realignment of the first and second band portions 100 and 150 of the wrist-wearable device 190 is illustrated in FIG. 1 at the second point in time 194.

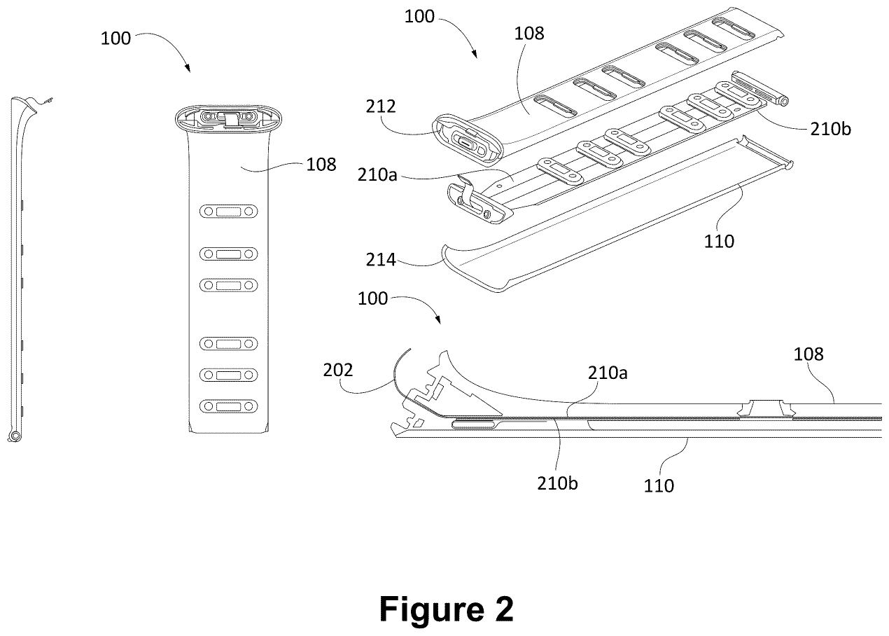

FIG. 2 illustrates an example method of manufacturing of a band section of a wrist-wearable device, in accordance with some embodiments. In particular, FIG. 2 shows the method of manufacturing the first band portion 100. Manufacturing each band section independent from other band sections (e.g., the second band section 150) and the capsule 120 portion allows for the formation of a modular wrist-wearable device 190. In some embodiments, the second band portion 150 can be manufactured in a similar manner.

FIG. 2 further illustrates the method of manufacturing including a two-shot cosmetic overmolded on an internal electronics stack 210. In some embodiments, the internal electronics stack 210 includes the first magnetic structure comprising the first magnetic portion 104a, the second magnetic portion 104b, and the third magnetic portion 104c, as well as one or more neuromuscular-signal sensors 106a-106f prior to overmolding, such that these components are encapsulated within the first and second shot portions 212 and 214 during the overmolding process. In some embodiments, the overmolded section comprises the magnetic structures. The first shot portion 212 is manufactured first by overmolding onto a top portion of the internal electronics stack 210a using a liquid injection molding (LIM) process, followed by the second shot portion 214 being overmolded onto a bottom portion of the internal electronics stack 210b using the same LIM process. The internal stack 210 is fully encapsulated inside of the first and second shot portions 212 and 214, with only areas dedicated to assembly/integration of sensors and integration to the capsule 120 portion exposed after overmolding. For example, the connection point 202 is exposed such that it can be connected to a capsule 120 at a later stage during manufacturing.

In some embodiments, the two-shot overmolding process utilizes liquid injection molding (LIM) as described above. However, other molding processes may also be employed in the manufacturing of the two-shot band. For example, compression molding represents an alternative manufacturing process that may be used in this application. In some aspects, the selection of a particular molding process may depend on factors such as the desired material properties, production volume, or specific geometric requirements of the band section.

After manufacturing, the first band section 100 thickness is less than 4 mm (e.g., 3.15 mm), which includes the thickness of the first shot portion 214 at less than 2 mm (e.g., 1.29 mm) and the second shot portion 214 at less than 1 mm (e.g., 0.7 mm). The second shot portion 214 thickness includes a wall thickness less than 1 mm (e.g., 0.5 mm) and a texture depth less than 0.5 mm (e.g., 0.2 mm). In some embodiments, the overmolded material is liquid silicone rubber (LSR) material.

The method of manufacturing the first band portion 100 creates an electronics-enabled band that can be validated separately from other parts of a wrist-wearable device 190. Including a modular band allows separate fabrication and validation from the rest of the device and allows a user to switch the band at a future time. Utilizing the LSR includes additional advantages such as flexibility, improved skin feel and chemical resistance, and a thinner band structure to maintain the thinness of the band.

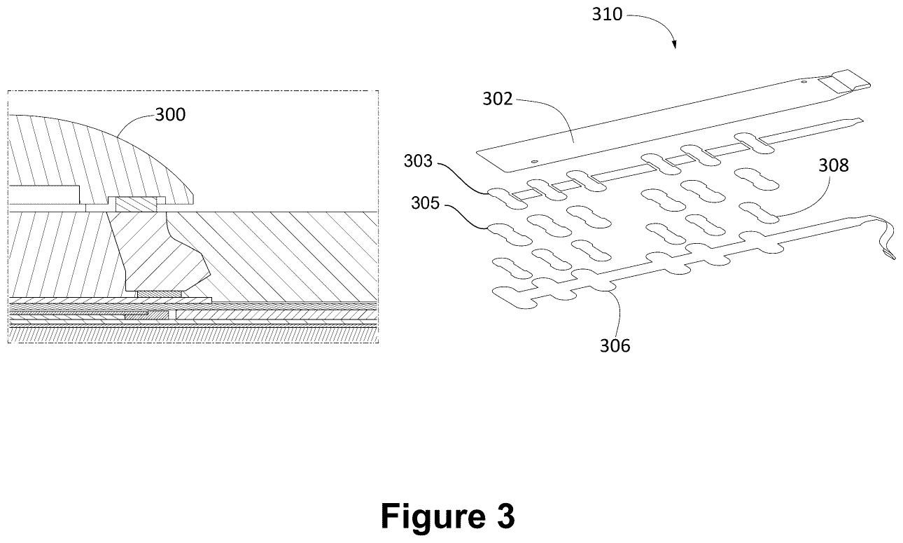

FIG. 3 illustrates an example embodiment integrating electronics into the band portion of a wrist-wearable device, in accordance with some embodiments. FIG. 3 illustrates a neuromuscular-signal sensor 300 (e.g., an instance of the one or more neuromuscular-signal sensors 106a-106f) integrated into the first band portion 100 of the wrist-wearable device 190 such that the assembly can survive daily wear by a user, be flexible, and be durable. In some embodiments, the neuromuscular-signal sensor 300 is an electromyography (EMG) sensor that is electrically and/or mechanically connected to a flexible printed circuit assembly (FPCA) 310 coupled to a strain relief layer 302 using one or more pressure-sensitive adhesives (PSAs) such that the flexible printed circuit (FPC) 306 remains flat. The different thicknesses of the one or more PSAs 308 allow the FPCA 310 to lie flat for better reliability and simplified assembly. In some embodiments, the FPCA 310 is a three-layer symmetrical stack-up that further reduces the strain when the wrist-wearable device 190 is bent (e.g., such as when the first and second band portions 100 and 150 are around the circumference of a user's wrist). When the FPCA 310 is fully flat, it brings the traces closer to a neutral axis of the first band section 100, which reduces strain on traces and prolongs the cycle bending life of the band section 100. In some embodiments, the strain relief layer includes a manufactured fiber that is spun from an LCP configured to reduce the stress seen when bending the first band section 100 and to protect the one or more neuromuscular-signal sensors 106a-106f coupled to the first band section 100. In some embodiments, the fully flat design is enabled by reducing one or more PSAs 308 (e.g., the stiffener) under the strain relief layer 302 to a minimum feasible thickness. In some embodiments, the stack up includes FPCA to PSAs 303 that couple the flexible printed circuit assembly 310 to the strain relief layer 302, and dogbone stiffeners comprising a flexible printed circuit (FPC) 305 that provide structural support while maintaining flexibility of the assembly. In some embodiments, the FPCA 310 is positioned within the internal electronics stack 210, located between the top portion of the internal electronics stack 210a and the bottom portion of the internal electronics stack 210b as shown in FIG. 2, such that the FPCA 310 becomes encapsulated within the first shot portion 212 and the second shot portion 214 during the overmolding process.

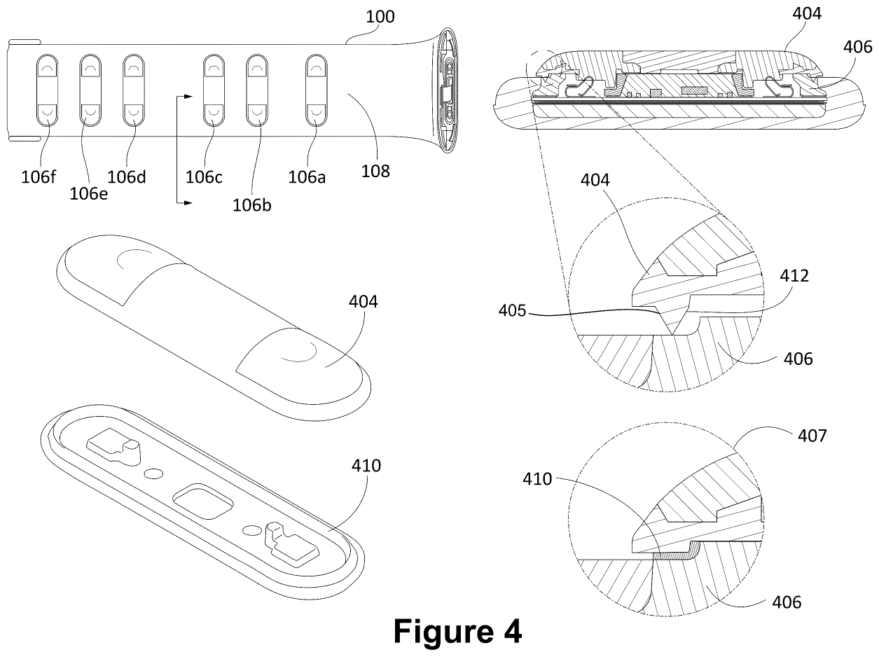

FIG. 4 illustrates an example method of coupling a neuromuscular-signal sensor to a band portion of a wrist-wearable device, in accordance with some embodiments. FIG. 4 illustrates an EMG sensor 404 coupled to the first band section 100 of the wrist-wearable device via ultrasonic welding. In some embodiments, EMG sensor 404 is an instance of one or more neuromuscular-signal sensors 106a-106f on a first side of the first band section 108. In some embodiments, the first band section 100 is manufactured first (e.g., as illustrated in FIG. 2) and subsequently the EMG sensor 404 is coupled to the first band section 100. The first band section 100 includes one or more receivers (e.g., receiver 406 is one example shown in FIG. 4) configured to receive the EMG sensor 404. In some embodiments, the receiver 406 is injection molded. The EMG sensor 404 is attached to the receiver 406 using ultrasonic welding, which includes melting the two thermoplastics of the EMG sensor 404 and the receiver 406 together via high-frequency vibration. To control the melted or bonding area, this design utilizes a 60-degree energy director 405 that melts into a step joint between the EMG sensor 404 and the receiver 406 (e.g., as shown in the close up 407). The EMG sensor 404 includes the first plastic component 412, and the perimeter of the EMG sensor 404 is the second plastic component. This second plastic component borders the perimeter of the EMG sensor 404, creating a 360-degree seal between the EMG sensor 404 and the receiver 406.

By coupling the EMG sensor 404 in the first band section 100 in a mechanically robust way, a sealed bond line (e.g., the perimeter 410) is created to protect the internal electronics and promote the reliable transmission of EMG signals. Furthermore, using ultrasonic welding creates a high mechanical retention of the EMG sensor 404 to the first band section 100 that can withstand high-stress situations such as rubbing, contact with water or sweat, repeatedly donning and doffing the wrist-wearable device 190, etc. The sealed design also allows for continued band function during specific-use cases (e.g., hand washing, rain, exercising, swimming). Additionally, a small footprint is required for this attachment method that enables an optimized form factor for the band. In some embodiments, in addition to ultrasonic welding, the EMG sensor 404 may be coupled to the first band section 100 using heat-activated film (HAF), liquid-dispensed adhesive (LDA), or a combination thereof.

Referring to FIG. 2, the bonding occurs at the interface between the first shot portion 212 and the second shot portion 214, where the overmolded liquid silicone rubber material encapsulates the internal electronics stack 210. In some embodiments, bonding may also occur at the connection point 202, which remains exposed after overmolding to allow subsequent attachment to the capsule 120 portion of the wrist-wearable device 190. The two-shot overmolding process creates a bond between the first shot portion 212 and the top portion of the internal electronics stack 210a, as well as between the second shot portion 214 and the bottom portion of the internal electronics stack 210b, thereby securing the internal components within the band structure.

Furthermore, as shown in FIG. 3, the electrical connection is established through the flexible printed circuit 306, which forms part of the flexible printed circuit assembly 310. In some embodiments, the electrical connection extends from the neuromuscular-signal sensor 300 through conductive traces within the flexible printed circuit 306 to enable signal transmission to processing components of the wrist-wearable device 190. The flexible printed circuit assembly 310 is coupled to the strain relief layer 302 using one or more pressure-sensitive adhesives, and the three-layer symmetrical stack-up of the FPCA 310 positions the conductive traces closer to a neutral axis of the first band section 100. In some aspects, this configuration may reduce strain on the electrical connections during bending and may prolong the operational life of the electrical pathways within the band section.

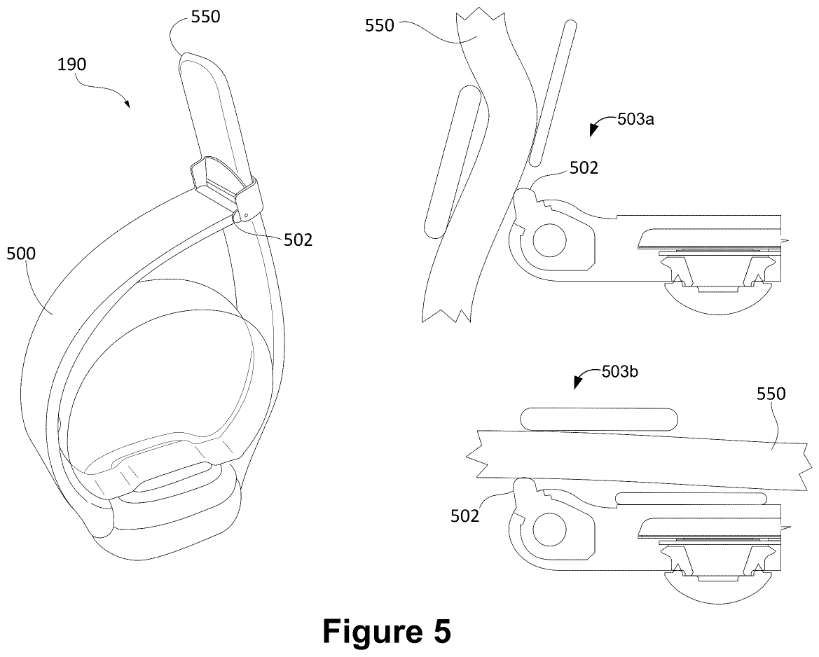

FIG. 5 illustrates an example embodiment of a band of the wrist-wearable device, in accordance with some embodiments. In some embodiments, the band closure method is designed to keep the band portions securely attached throughout the donning and doffing process. In some embodiments, the first band portion 500 and the second band portion 550 shown in FIG. 5 correspond to the first band section 100 and the second band section 150 described in FIG. 1. The band closure mechanism 502 may be used in conjunction with the magnetic coupling described in FIG. 1 to provide additional retention, and the first band portion 500 may be manufactured using the two-shot overmolding process illustrated in FIG. 2. A first band portion 500 can include electrodes (e.g., neuromuscular-signal sensors 106a-106f) and the band closure mechanism described herein can maintain tension after donning for all-day wear to enable low electrode contact loss for EMG performance. The band closure includes a band closure mechanism 502 that is configured to receive the second band portion 550 and adjust to be in an unlocked state (e.g., a kinked state) or a locked state. While the band closure mechanism 502 is in an unlocked state 503a, the second band portion 550 can slide through the band closure mechanism 502 with some resistance. While the band closure mechanism 502 is in a closed (e.g., locked) state 503b, the second band portion 550 is locked into position and cannot slide through the band closure mechanism 502. In accordance with a determination that the user wants to remove the wrist-wearable device 190, the band closure mechanism 502 is placed into an unlocked state and to remove the second band portion 550 from the adjustment mechanism 502. In another example, when the user wants to put on the wrist-wearable device 190 and maintain the first band portion 500 and the second band portion 550 in place, the band closure mechanism 502 can be put into the locked state where the circumference of the wrist-wearable device 190 cannot be changed without adjusting the band closure mechanism 502.

Furthermore, the band closure mechanism 502 is easy to lock and provides tactile feedback to adjust. The band closure mechanism 502 holds tension when locked but maintains a level of comfort for the user. The band closure mechanism 502 is adjustable such that the wrist-wearable device 190 can fit on any wrist size and use elastomer bands without specific indexed positions. Additionally, the band closure mechanism 502 is able to hold tension between the first band portion 500 and the second band portion 550 when transitioning between an unlocked and a locked state.

In some embodiments, the band closure mechanism 502 may include a plurality of discrete indexed positions arranged along the second band portion 550. The indexed positions may be configured to provide a ratcheting feel for the user as the second band portion 550 is drawn through the band closure mechanism 502 during donning and doffing of the wrist-wearable device 190. In some aspects, the ratcheting configuration may provide audible and/or tactile feedback to the user as the band closure mechanism 502 engages with successive indexed positions, thereby allowing the user to perceive incremental adjustments to the band circumference. The indexed positions may correspond to predetermined circumference settings that accommodate a range of wrist sizes while maintaining consistent tension on the neuromuscular-signal sensors 106a-106f for reliable EMG signal detection. In some cases, the ratcheting mechanism may facilitate one-handed operation, allowing the user to tighten the band by pulling the second band portion 550 through the band closure mechanism 502 until a desired fit is achieved.

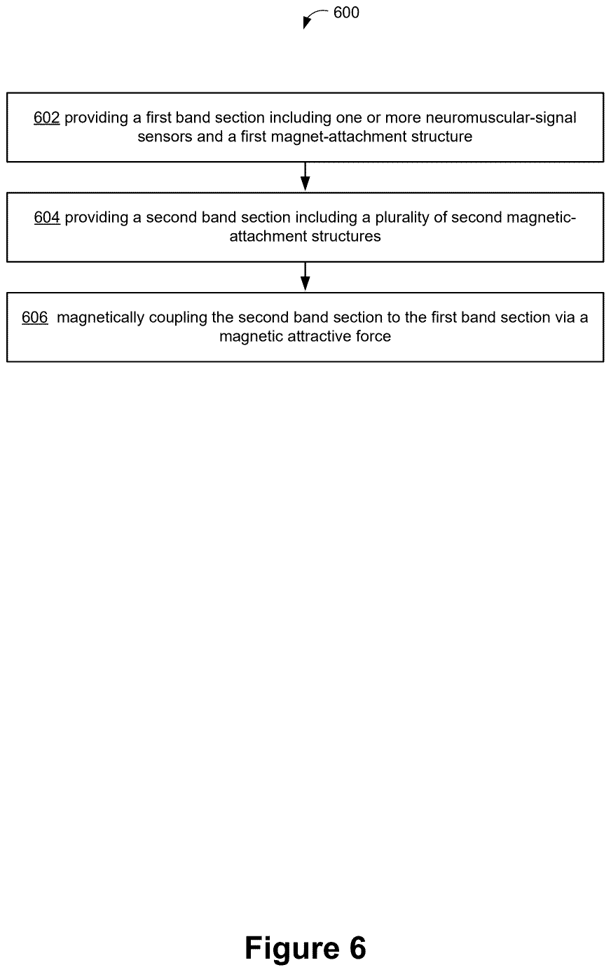

FIG. 6 shows an example method flow chart for coupling a first band section to a second band section of a wrist-wearable device, in accordance with some embodiments. In some embodiments, the various operations of the methods described herein are interchangeable and/or optional, and respective performance of the methods can be performed by any of the aforementioned devices, systems, or combination of devices and/or systems.

FIG. 6 further illustrates a method of coupling the second band section to the first band section of the wrist-wearable device. Each respective band section includes magnetic portions with polarities opposite of the magnetic portions on the band section they will couple such that the two band portions attract to each other. In some embodiments, including multiple magnetic portions of alternating polarity also provides a self-alignment feature such that if the band sections are placed on each other but are unaligned, the polarity of magnets will realign the band. (A1) FIG. 6 shows a flow chart of a method 600 for coupling the second band section to the first band section of the wrist-wearable device via magnetic properties, in accordance with some embodiments.

The method 600 occurs at a wrist-wearable device (e.g., wrist-wearable device 190) with one or more band sections and a capsule 120 portion. In some embodiments, the method 600 includes (602) providing a first band section (e.g., first band section 100) including one or more neuromuscular-signal sensors (e.g., one or more neuromuscular-signal sensors 106a-106f) and a first magnet-attachment structure (e.g., including the first magnetic portion 104a, the second magnetic portion 104b, and the third magnetic portion 104c). The neuromuscular-signal sensors configured to be in contact with a wrist of a user on a first side of the first band section (e.g., first side of the first band section 108) and the first magnetic-attachment structure having a first magnetic portion (e.g., third magnetic portion 104c) with a first magnetic polarity in between a second magnetic portion (e.g., the first magnetic portion 104a and/or the second magnetic portion 104b) having a second magnetic polarity, the first magnetic-attachment structure being on a second side of the first band section (e.g., the second side of the first band section 110) opposite the first side of the first band section (e.g., the first side of the first band section 108). As described above and illustrated in FIG. 1, the first side of the first band section 108 is coupled to the wrist of the user, and the second side of the second band section 110 is configured to couple with the second band section 150.

The method 600 further includes (604) providing a second band section (e.g., second band section 150) including a plurality of second magnetic-attachment structures (e.g., magnetic-attachment structures 154a-154e). The second band section (i) does not include neuromuscular-signal sensors and (ii) each of the plurality of second magnetic-attachment structures has a third magnetic portion (e.g., a third magnetic portion 156c) with the second magnetic polarity placed in between a fourth magnetic portion (e.g., the first magnetic portion 156a and/or the second magnetic portion 156b) with the first magnetic polarity. The second band section 150 and the plurality of second magnetic-attachment structures are described above and illustrated in FIG. 1.

The method 600 further includes (606) coupling the second band section (e.g., second band section 150) magnetically to the first band section (e.g., first band section 100) via a magnetic attractive force. The second band section is configured to magnetically couple with the first band section by way of (i) a magnetic attractive force between the first magnetic portion of the first band section (e.g., third magnetic portion 104c) and respective third magnetic portions (e.g., a third magnetic portion 156c) of the plurality of second magnetic-attachment structures, and (ii) a magnetic attractive force between the second magnetic portion (e.g., the first magnetic portion 104a and/or the second magnetic portion 104b) of the first band section and respective fourth magnetic portions (e.g., the first magnetic portion 156a and/or the second magnetic portion 156b) of the plurality of second magnetic-attachment structures. FIG. 1 describes and illustrates the first band section 100 and the second band section 150 coupled together via magnetic attractive force.

(A2) In some embodiments of A1 (described in reference to FIG. 6), the second band section is configured to magnetically repel the first band section while the band is in a first position. The first position includes at least one of (i) the first magnetic portion of the first band section (e.g., the third magnetic portion 104c) and the respective fourth magnetic portion of the plurality of second magnetic-attachment structures (e.g., the first magnetic portion 156a or the second magnetic portion 156b of magnetic-attachment structure 154e) are positioned such that there is a magnetic repulsive force therebetween to cause the band to adjust to a self-aligned position or (ii) the second magnetic portion (e.g., first magnetic portion 104a or the second magnetic portion 104b) of the first band section and respective third magnetic portions of the plurality of second magnetic-attachment structures (e.g., third magnetic portion 156c) are positioned such that there is a magnetic repulsive force therebetween to cause the band to adjust to the self-aligned position.

As described in FIG. 1, the wrist-wearable device 190 at a first point in time 192 illustrates that the first band section 100 and the second band section 150 are misaligned. Due to the repulsive forces of the magnetic structures contained inside of the first and second band portions 100 and 150, the magnetic structures are configured to automatically align when the magnetic portions (e.g., third magnetic portion 104c) of the first band section 100 magnetically attract to the magnetic portions (e.g., a third magnetic portion 156c) of the second band section 150. The wrist-wearable device 190 at the second point in time 194 illustrates the wrist-wearable device 190 after the first and second band sections 100 and 150 automatically align.

(A3) In some embodiments of any one of A1-A2, the plurality of second magnetic-attachment structures includes at least two magnetic sections (e.g., magnetic-attachment structure 154a and magnetic-attachment structure 154b) wherein the two magnetic sections are a predetermined distance away from each other. As illustrated and described above in FIG. 1, the separation distance between the magnetic sections is predetermined to allow for different sizing of the wrist-wearable device 190 such that it is more comfortable for the user and/or fits a larger wrist of the user.

(A4) In some embodiments of any one of A1-A3, the first magnetic-attachment structure (e.g., including the first magnetic portion 104a, the second magnetic portion 104b, and the third magnetic portion 104c) is continuous along a length of the first band section (e.g., first band section 100). As described above and illustrated in FIG. 1, the first magnetic-attachment structure comprises the first, second, and third magnetic portions 104a, 104b, and 104c and is continuous along the length of the first band section 100. This allows the second band portion 150 to couple to the first band section 100 along any point where the first magnetic-attachment structure is present.

(A5) In some embodiments of any one of A1-A4, the wrist-wearable device further includes a closure mechanism (e.g., the band closure mechanism 502) coupled to the first band section (e.g., first band section 100). While the closure mechanism is in the locked position, the second band portion cannot pass through the closure mechanism, and while the closure mechanism is in the unlocked position, the second band portion can pass through the closure mechanism while tension is maintained on the second band portion. As described above and illustrated in FIG. 5, the band closure mechanism 502 allows the user to maintain tension on the second band portion 150 while adjusting the tightness of the first and second band sections 100 and 150.

(A6) In some embodiments of any one of A1-A5, the wrist-wearable device further includes a capsule portion (e.g., capsule 120) with display configured to process one or more detected neuromuscular signals, and based on the one or more detected neuromuscular signals (i) determines one or more gestures performed by the user and (ii) displays one or more actions associated with the one or more gestures. In some embodiments, a user performs a gesture and one or more neuromuscular signals are detected at the neuromuscular-signal sensors. The capsule 120 portion of the wrist-wearable device can display one or more actions based on the performed gesture.

(A7) In some embodiments of any one of A1-A6, the first band section further includes (i) a layer including a manufactured fiber that is spun from an LCP (e.g., to strain relief layer 302) configured to provide stiffness for the first band section and (ii) one or more receivers (e.g., receiver 406) configured to couple to each respective neuromuscular-signal sensor. As described above and illustrated in FIGS. 3 and 4, the strain relief layer 302 includes stiffeners to support the receivers (e.g., receiver 406) and ultimately the assemblies for the neuromuscular-signal sensors after they are coupled to the first band portion 100. This ensures the first band portion 100 is more reliable.

(A8) In some embodiments of any one of A1-A7, each neuromuscular-signal sensor (e.g., one or more neuromuscular-signal sensors 106a-106f) is configured to be coupled with each respective receiver (e.g., receiver 406) via ultrasonic welding. As described in FIG. 4, each respective neuromuscular-signal sensor is coupled to a receiver 406 such that the sensors are attached to the first band section 100. This allows the neuromuscular-signal sensor 106a-106f assemblies to be manufactured independently of the first band section 100 and attached after they are both manufactured.

(A9) In some embodiments of any one of A1-A8, the second band section is configured to magnetically couple with the first band in a plurality of locations along the first band such that a circumference of the wrist-wearable device band is adjustable to a plurality of wrist sizes. As described above and shown in FIG. 1, the second band section 150 can couple to the first band section anywhere along the magnetic-attachment structure such that the wrist-wearable device can accommodate a small wrist size and/or a large wrist size.

(A10) In some embodiments of any of A1-A9, the second band section is configured to self-align when coupled with the first band section. As described above and illustrated in FIG. 1, the plurality of magnetic sections with opposite polarities on the magnetic-attachment structures are configured such that if the first and second band sections are coupled to one another but misaligned, they will automatically adjust and align to satisfy the magnetic attraction forces.

(B1) In accordance with some embodiments, a method of manufacturing a wrist-wearable device includes overmolding a first portion of a band section onto a first side of one or more electronics of the wrist-wearable device, overmolding a second portion of the band section onto a second side, opposite the first side, of the one or more electronics, and coupling, via ultrasonic welding, at least one or more neuromuscular-signal sensors to the one or more electronics.

(C1) In accordance with some embodiments, a system includes an extended-reality headset configured to display an extended-reality environment and a wrist-wearable device communicatively coupled to the extended-reality device. The wrist-wearable device is configured to receive input commands for interacting with the extended-reality environment, and the wrist-wearable device is configured in accordance with any one of A1-A10.

(D1) In accordance with some embodiments, a method of operating a wrist-wearable device includes (i) receiving data from the wrist-wearable device indicating that an input command (e.g., an EMG-based input command) is received and (ii) providing data about the input command to an extended-reality headset. The method further includes that the wrist-wearable device is configured in accordance with any one of A1-A10.

(E1) In accordance with some embodiments, a method of operating a wrist-wearable device includes a non-transitory computer-readable storage medium including instructions that, when executed by a system that includes a wrist-wearable device that is in communication with an extended-reality headset, cause the system to (i) receive data indicating an input command from the wrist-wearable device has been received and (ii) provide data about the input command to the extended-reality headset for interacting with an extended-reality environment. The wrist-wearable device is configured in accordance with any one of A1-A10.

(F1) In accordance with some embodiments, a wrist-wearable device including a band for sensing neuromuscular signals includes a first band section, a second band section, a plurality of neuromuscular-signal sensors disposed within the first band section, where the neuromuscular-signal sensors are configured to be in contact with a wrist of a user on a first side of the first band section, a first attachment magnet disposed within the first band section where the first attachment magnet has a first central magnetic portion with a first magnetic polarity disposed between a plurality of first lateral magnetic portions each having a second magnetic polarity, and a plurality of second attachment magnets disposed within the second band section, where each respective second attachment magnet has a second central magnetic portion with the second magnetic polarity disposed between a plurality of second lateral magnetic portions each having the first magnetic polarity, where a magnetic attractive force between the first central magnetic portion of the first attachment magnet and respective second central magnetic portions of the plurality of second attachment magnets and the magnetic attractive force between the plurality of first lateral magnetic portions of the first attachment magnet and respective plurality of second lateral magnetic portions of the plurality of second attachment magnets are configured to magnetically couple the second band section with the first band section.

For example, as shown in FIG. 1, the wrist-wearable device 190 includes a first band section 100 and a second band section 150 configured to magnetically couple together. The first band section 100 includes neuromuscular-signal sensors 106a-106f disposed on a first side of the first band section 108 that is configured to contact the wrist of a user. A first attachment magnet is disposed within the first band section 100 on a second side of the first band section 110, where the first attachment magnet has a third magnetic portion 104c with a first magnetic polarity positioned between a first magnetic portion 104a and a second magnetic portion 104b each having a second magnetic polarity. The second band section 150 includes a plurality of second attachment magnets, such as magnetic-attachment structures 154a-154e, where each respective second attachment magnet has a third magnetic portion 156c with the second magnetic polarity disposed between a first magnetic portion 156a and a second magnetic portion 156b each having the first magnetic polarity. The magnetic attractive force between the third magnetic portion 104c of the first attachment magnet and respective third magnetic portions 156c of the plurality of second attachment magnets, combined with the magnetic attractive force between the first magnetic portion 104a and second magnetic portion 104b of the first attachment magnet and respective first magnetic portions 156a and second magnetic portions 156b of the plurality of second attachment magnets, are configured to magnetically couple the second band section 150 with the first band section 100, as illustrated at the second point in time 194 where the band sections are properly aligned.

(F2) In some embodiments of F1, the second band section is configured to magnetically repel the first band section while the band is in a first position, the first position including the first central magnetic portion of the first band section and the plurality of second lateral magnetic portions of the plurality of second attachment magnets are positioned such that there is a magnetic repulsive force therebetween to cause the band to adjust to a self-aligned position, or the second central magnetic portion of each respective second attachment magnet and respective first lateral magnetic portions of the first attachment magnet are positioned such that there is a magnetic repulsive force therebetween to cause the band to adjust to the self-aligned position.

For example, as shown in FIG. 1 at the first point in time 192, when the first band section 100 and the second band section 150 are initially brought together in a misaligned configuration, the third magnetic portion 104c of the first attachment magnet may be positioned adjacent to the first magnetic portion 156a or the second magnetic portion 156b of one of the magnetic-attachment structures 154a-154e. In this misaligned position, both portions have the same magnetic polarity, creating a magnetic repulsive force between them. Similarly, if the first magnetic portion 104a of the first band section 100 is positioned adjacent to the third magnetic portion 156c of magnetic-attachment structure 154e during an initial coupling attempt, a magnetic repulsive force occurs because both portions share the same magnetic polarity. These repulsive forces cause the band sections to automatically shift relative to one another until the opposing polarity portions align properly, as illustrated at the second point in time 194 where the third magnetic portion 104c aligns with the third magnetic portion 156c of the corresponding magnetic-attachment structure, and the first magnetic portion 104a aligns with either the first magnetic portion 156a or the second magnetic portion 156b.

In some cases, as shown in FIG. 1, when the second band section 150 is placed over the first band section 100 at an angle or with lateral offset, multiple repulsive forces may act simultaneously between misaligned magnetic portions. For instance, the third magnetic portion 104c may repel the second magnetic portion 156b of magnetic-attachment structure 154c while the second magnetic portion 104b may repel the third magnetic portion 156c of magnetic-attachment structure 154d. These combined repulsive forces work together to guide the band sections toward the self-aligned position shown at the second point in time 194, where all magnetic portions with opposite polarities are properly aligned to create attractive forces that secure the band sections together.

(F3) In some embodiments of any of F1-F2, the plurality of second lateral magnetic portions of each respective second attachment magnet are spaced apart from each other.

For example, as shown in FIG. 1, the plurality of second attachment magnets includes magnetic-attachment structures 154a, 154b, 154c, 154d, and 154e that are spaced apart from each other along the length of the second band section 150. The spacing between adjacent magnetic-attachment structures allows the second band section 150 to flex and conform to different wrist sizes while maintaining magnetic coupling capability with the first band section 100. The predetermined spacing between the magnetic-attachment structures 154a-154e is configured to provide even distribution of force when the wrist-wearable device 190 is worn, thereby ensuring that the neuromuscular-signal sensors 106a-106f on the first band section 100 maintain consistent contact with the skin of the user.

(F4) In some embodiments of any of F1-F3, the first attachment magnet is a continuous elongated strip along a length of the first band section.

For example, as shown in FIG. 1, the first attachment magnet extends continuously along the longitudinal axis of the first band section 100, spanning from a region near the first connection point 102 to an opposite end of the first band section 100. The continuous configuration of the first attachment magnet, which includes the first magnetic portion 104a, the third magnetic portion 104c, and the second magnetic portion 104b arranged in sequence, allows the second band section 150 to magnetically couple at any position along the length of the first band section 100. In some cases, the continuous elongated strip configuration may enable the wrist-wearable device 190 to accommodate a wide range of wrist circumferences by allowing the magnetic-attachment structures 154a-154e of the second band section 150 to engage with the first attachment magnet at different positions along the first band section 100, as illustrated by the adjustable coupling shown in FIG. 1.

(F5) In some embodiments of any of F1-F4, the first attachment magnet comprises a magnetic rubber material formed from liquid silicone rubber mixed with magnetic particles.

For example, as shown in FIG. 2, the first attachment magnet may be formed from a magnetic rubber material comprising liquid silicone rubber mixed with magnetic particles in a predetermined ratio to create a homogenous blend. The magnetic rubber material may be compression molded into a shape matching the first band section 100 and permanently magnetized using a uni-directional field to ensure stronger magnetic force in the direction of tail retention. In some cases, the inclusion of magnetic particles in the liquid silicone rubber may not substantially alter the material properties of the base rubber material, thereby maintaining flexibility and durability of the first band section 100 while providing sufficient magnetic coupling force with the second band section 150.

(F6) In some embodiments of any of F1-F5, the plurality of second attachment magnets are coupled by a connection layer comprising a strain relief layer formed of a manufactured fiber spun from a liquid crystal polymer.

For example, as shown in FIG. 1, the plurality of second attachment magnets, including magnetic-attachment structures 154a-154e, may be linked by a connection layer 158 that provides structural support while allowing flexibility of the second band section 150. The connection layer 158 may comprise a strain relief layer formed of a manufactured fiber spun from a liquid crystal polymer, which may reduce mechanical stress on the magnetic-attachment structures 154a-154e during bending and flexing of the second band section 150. In some cases, the connection layer 158 may maintain electrical connectivity between components while distributing tensile forces along the length of the second band section 150, thereby prolonging the operational life of the wrist-wearable device 190.

(F7) In some embodiments of any of F1-F6, the second band section further includes an end portion having magnetic properties configured to couple to the first band section.

For example, as shown in FIG. 1, the second band section 150 includes a tail portion 159 that extends beyond the magnetic-attachment structures 154a-154e and is configured to couple to the first band section 100 when the wrist-wearable device 190 is worn. The tail portion 159 may include magnetic properties that allow it to magnetically attach to the second side of the first band section 110, thereby preventing the excess length of the second band section 150 from extending away from the wrist during use. In some cases, the tail portion 159 may automatically lie flat along the first band section 100 due to magnetic attraction between the tail portion 159 and the first attachment magnet disposed within the first band section 100, as illustrated in the assembled configuration of the wrist-wearable device 190.

(F8) In some embodiments of F7, the end portion of the second band section includes stronger magnetic properties than other portions of the second band section such that additional force is required to remove the end portion from the first band section.

For example, as shown in FIG. 1, the tail portion 159 may include one or more magnetized segments with enhanced magnetic field strength compared to the magnetic-attachment structures 154a-154e positioned along the main body of the second band section 150. The stronger magnetic properties of the tail portion 159 may be achieved through increased magnetic particle density or larger magnetic elements within the tail portion 159, ensuring high retention force when the tail portion 159 is coupled to the first band section 100. In some cases, the enhanced magnetic coupling between the tail portion 159 and the first attachment magnet disposed within the first band section 100 may require deliberate user action to disengage, thereby preventing accidental separation during active motion or daily wear of the wrist-wearable device 190. The stronger magnetic properties may ensure that the excess tail stays retained and lies substantially flat along the first band section 100 during use, as illustrated in the assembled configuration shown in FIG. 1.

(F9) In some embodiments of any of F1-F8, the first band section is connected to a first side of a capsule via a first connection point, and the second band section is connected to a second side of the capsule, opposite the first side of the capsule, via a second connection point.

For example, as shown in FIG. 1, the first band section 100 is connected to the first side of capsule 101 via the first connection point 102, and the second band section 150 is connected to the second side of capsule 151 via the second connection point 152. The capsule 120 may serve as a computing system of the wrist-wearable device 190 and may house processing components, display elements, and power systems for operating the device. In some cases, the first connection point 102 and the second connection point 152 may provide both mechanical attachment and electrical connectivity between the respective band sections and the capsule 120, allowing sensor data from the neuromuscular-signal sensors 106a-106f to be transmitted to processing components within the capsule 120. As illustrated in FIG. 2, the connection point 202 may remain exposed after the overmolding process to facilitate subsequent attachment to the capsule 120 portion of the wrist-wearable device.

(F10) In some embodiments of any of F1-F9, the wrist-wearable device further includes a capsule portion with a display configured to process one or more detected neuromuscular signals and based on the one or more detected neuromuscular signals determine one or more gestures performed by the user, and display one or more actions associated with the one or more gestures on the display.

For example, as shown in FIG. 1, the capsule 120 may include processing circuitry configured to analyze neuromuscular signals detected by the neuromuscular-signal sensors 106a-106f positioned along the first band section 100. The capsule 120 may determine that a specific pattern of muscle activation corresponds to a hand gesture such as a pinch or swipe motion, and may display corresponding visual feedback or execute associated commands on its display. In some cases, as illustrated in the wrist-wearable device 190 configuration, the capsule 120 may communicate with external devices to trigger actions in response to detected gestures, such as controlling smart home devices or navigating user interfaces. The processing capabilities of the capsule 120 may enable real-time gesture recognition and response, allowing users to interact with digital environments through natural muscle movements detected by the sensors 106a-106f.

(F11) In some embodiments of any of F1-F10, the second band section is configured to magnetically couple with the first band section in a plurality of locations along the first band section such that a circumference of the band is adjustable to a plurality of wrist sizes.

For example, as shown in FIG. 1, the continuous elongated strip configuration of the first attachment magnet extending along the first band section 100 allows the magnetic-attachment structures 154a-154e of the second band section 150 to engage at multiple positions along the length of the first band section 100. The adjustable coupling capability may accommodate wrist circumferences ranging from small to large sizes by allowing a user to position any of the magnetic-attachment structures 154a-154e at different locations along the first attachment magnet. In some cases, the spacing between the magnetic-attachment structures 154a-154e may provide discrete adjustment increments, while the continuous nature of the first attachment magnet allows for fine-tuning of the fit between these increments, as illustrated by the assembled configuration of the wrist-wearable device 190 shown in FIG. 1.

(F12) In some embodiments of any of F1-F11, the second band section is configured to self-align when coupled with the first band section.

For example, as shown in FIG. 1 at the first point in time 192, when the first band section 100 and the second band section 150 are initially brought together in a misaligned configuration, the opposing magnetic polarities of the first attachment magnet and the plurality of second attachment magnets create repulsive forces that automatically reposition the band sections relative to one another. The self-alignment mechanism may cause the third magnetic portion 104c of the first band section 100 to align with the third magnetic portion 156c of a corresponding magnetic-attachment structure from the plurality of structures 154a-154e, while simultaneously aligning the first magnetic portion 104a and the second magnetic portion 104b with respective second magnetic portions 156b and first magnetic portions 156a. As illustrated at the second point in time 194 in FIG. 1, the self-aligned configuration ensures that the neuromuscular-signal sensors 106a-106f maintain proper contact with the user's wrist without requiring manual adjustment by the user.

(F13) In some embodiments of any of F1-F12, the second band section does not include neuromuscular-signal sensors.

For example, as shown in FIG. 1, the second band section 150 may be configured without neuromuscular-signal sensors, allowing the second band section 150 to serve primarily as a retention and adjustment mechanism for securing the wrist-wearable device 190 around a user's wrist. The absence of sensors in the second band section 150 may reduce manufacturing complexity and cost while concentrating all sensing functionality within the first band section 100, where the neuromuscular-signal sensors 106a-106f are positioned to maintain consistent contact with the user's skin. In some cases, as illustrated in FIG. 1, the second band section 150 may include only the plurality of magnetic-attachment structures 154a-154e, the connection layer 158, and the tail portion 159, thereby providing a streamlined design that focuses on secure magnetic coupling with the first band section 100 without the need for additional electronic components or sensor integration.

(G1) In accordance with some embodiments, a system comprises a wrist-wearable device including a band comprising a first band section, a second band section, a plurality of neuromuscular-signal sensors disposed within the first band section, where the neuromuscular-signal sensors are configured to be in contact with a wrist of a user on a first side of the first band section, a first attachment magnet disposed within the first band section where the first attachment magnet has a first central magnetic portion with a first magnetic polarity disposed between a plurality of first lateral magnetic portions each having a second magnetic polarity, and a plurality of second attachment magnets disposed within the second band section, where each respective second attachment magnet has a second central magnetic portion with the second magnetic polarity placed disposed between a plurality of second lateral magnetic portions each having the first magnetic polarity, where a magnetic attractive force between the first central magnetic portion of the first attachment magnet and respective second central magnetic portions of the plurality of second attachment magnets and the magnetic attractive force between the plurality of first lateral magnetic portions of the first attachment magnet and respective plurality of second lateral magnetic portions of the plurality of second attachment magnets are configured to magnetically couple the second band section with the first band section.

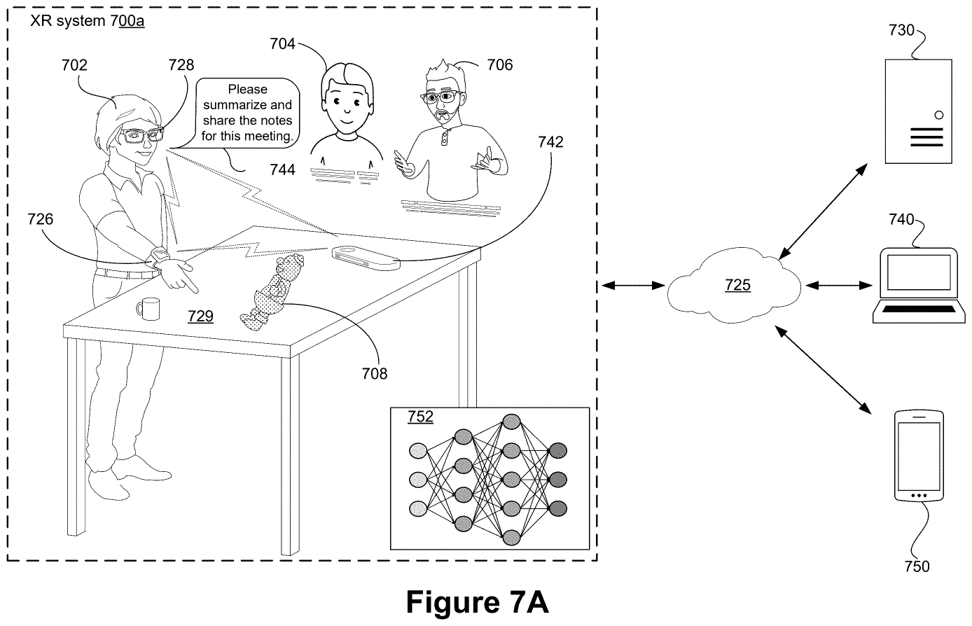

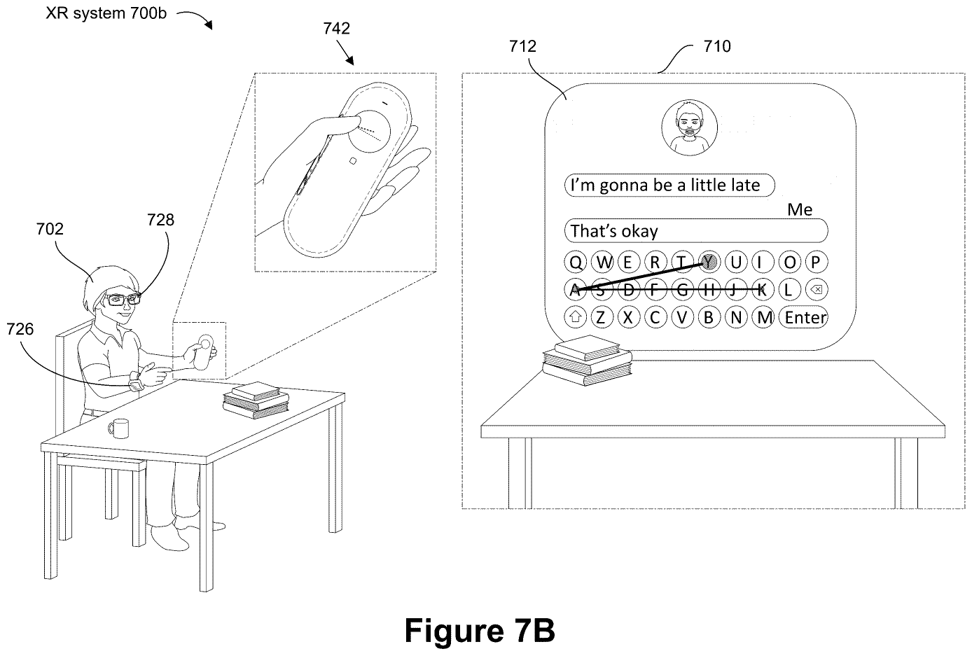

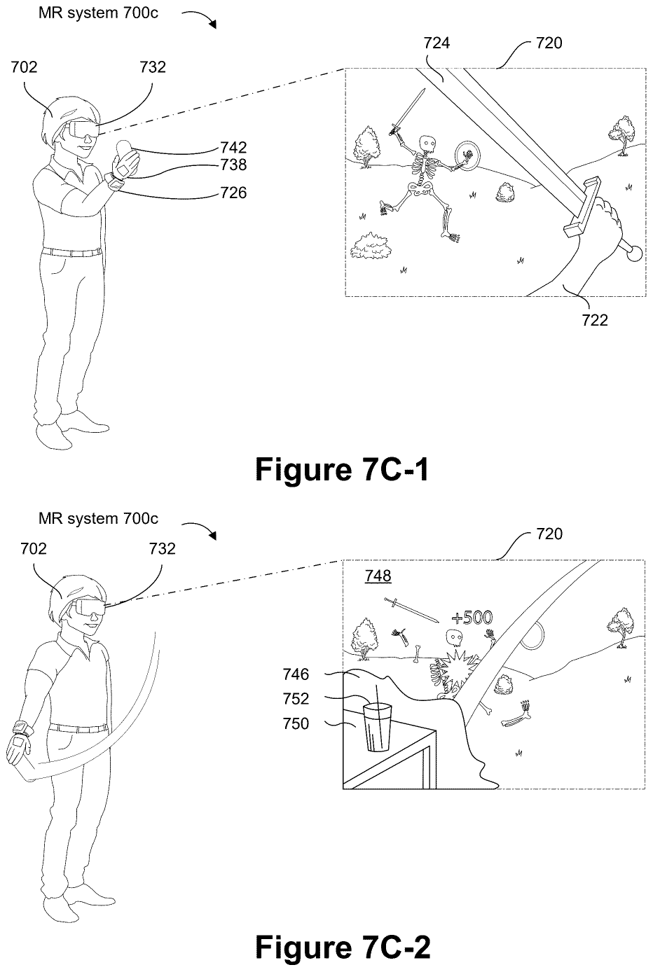

For example, as shown in FIG. 7A, the system may include an XR system 700a that incorporates the wrist-wearable device 726 as part of an integrated extended reality environment. The wrist-wearable device 726 may include the first band section 100 and the second band section 150 configured to magnetically couple together as described in FIG. 1. The system may further include the AR device 728 and the HIPD 742, which may communicate with the wrist-wearable device 726 via the network 725 to provide coordinated user interactions within the extended reality environment. In some cases, the neuromuscular-signal sensors 106a-106f disposed within the first band section 100 may detect muscle activation patterns that are transmitted to the capsule 120 and subsequently shared with other components of the system, such as the server 730 or the computer 740, to enable gesture-based control of virtual content including the avatar 704, the digital representation of contact 706, and the virtual object 708 displayed within the user's field of view.

(G2) In some embodiments of G1, the plurality of second lateral magnetic portions of each respective second attachment magnet are a predetermined distance away from each other.

For example, as shown in FIG. 1, the plurality of second attachment magnets may include magnetic-attachment structures 154a and 154b that are spaced apart by a predetermined distance along the length of the second band section 150. The predetermined spacing between adjacent magnetic-attachment structures may allow the second band section 150 to flex and conform to different wrist sizes while maintaining magnetic coupling capability with the first band section 100. In some cases, the spacing between the magnetic-attachment structures 154a-154e may be configured to provide even distribution of force when the wrist-wearable device 190 is worn, thereby ensuring that the neuromuscular-signal sensors 106a-106f on the first band section 100 maintain consistent contact with the skin of the user during operation within the system.

(G3) In some embodiments of any of G1-G2, the first attachment magnet is continuous along a length of the first band section.

For example, as shown in FIG. 1, the first attachment magnet may extend continuously along the longitudinal axis of the first band section 100, spanning from a region near the first connection point 102 to an opposite end of the first band section 100. The continuous configuration of the first attachment magnet, which includes the first magnetic portion 104a, the third magnetic portion 104c, and the second magnetic portion 104b arranged in sequence, may allow the second band section 150 to magnetically couple at any position along the length of the first band section 100. In some cases, the continuous elongated strip configuration may enable the wrist-wearable device 726 within the system to accommodate a wide range of wrist circumferences by allowing the magnetic-attachment structures 154a-154e of the second band section 150 to engage with the first attachment magnet at different positions along the first band section 100.

(G4) In some embodiments of any of G1-G3, the wrist-wearable device further includes a closure mechanism coupled to the first band section such that while the closure mechanism is in a locked position, the second band section cannot pass through the closure mechanism, and while the closure mechanism is in an unlocked position, the second band section can pass through the closure mechanism while tension is maintained on the second band section.

For example, as shown in FIG. 5, the wrist-wearable device 190 may include the band closure mechanism 502 coupled to the first band portion 500, where the band closure mechanism 502 is configured to receive the second band portion 550 and adjust between an unlocked state and a locked state. While the band closure mechanism 502 is in the locked state, the second band portion 550 may be secured in position and cannot slide through the band closure mechanism 502, thereby maintaining a fixed circumference for the wrist-wearable device 190. In some cases, while the band closure mechanism 502 is in the unlocked state, the second band portion 550 may slide through the band closure mechanism 502 with some resistance while tension is maintained on the second band portion 550, allowing the user 702 to adjust the fit of the wrist-wearable device 726 within the system shown in FIG. 7A without completely removing the device from their wrist.

(G5) In some embodiments of any of G1-G4, the wrist-wearable device further includes a capsule portion with display configured to process one or more detected neuromuscular signals and based on the one or more detected neuromuscular signals determine one or more gestures performed by the user, and display one or more actions associated with the one or more gestures.

For example, as shown in FIG. 1, the capsule 120 may include processing circuitry and a display configured to analyze neuromuscular signals detected by the neuromuscular-signal sensors 106a-106f positioned along the first band section 100. The capsule 120 may determine that a specific pattern of muscle activation corresponds to a hand gesture such as a pinch or swipe motion, and may display corresponding visual feedback or execute associated commands on its display. In some cases, as illustrated in the system configuration shown in FIG. 7A, the capsule 120 of the wrist-wearable device 726 may communicate with the AR device 728 and the HIPD 742 via the network 725 to trigger actions in response to detected gestures, such as manipulating the virtual object 708 or interacting with the avatar 704 and the digital representation of contact 706 within the extended reality environment.

(G6) In some embodiments of any of G1-G5, the first band section further includes a layer including a manufactured fiber that is spun from an LCP configured to provide stiffness for the first band section, and one or more receivers configured to couple to each respective neuromuscular-signal sensor.

For example, as shown in FIG. 3, the first band section may include the strain relief layer 302 comprising a manufactured fiber spun from a liquid crystal polymer, which may be configured to provide structural stiffness for the first band section while maintaining flexibility during bending. As illustrated in FIG. 4, the first band section 100 may further include one or more receivers 406 configured to couple to each respective neuromuscular-signal sensor from the sensors 106a-106f. In some cases, the strain relief layer 302 may reduce mechanical stress on the flexible printed circuit assembly 310 and the neuromuscular-signal sensors during flexing of the first band section, thereby prolonging the operational life of the wrist-wearable device 726 within the system shown in FIG. 7A.

(G7) In some embodiments of any of G1-G6, each neuromuscular-signal sensor is configured to couple with each respective receiver via ultrasonic welding.

For example, as shown in FIG. 4, each neuromuscular-signal sensor from the sensors 106a-106f may be configured to couple with a respective receiver 406 via ultrasonic welding, which includes melting thermoplastic components of the electrode pill 410 and the receiver 406 together via high-frequency vibration. The ultrasonic welding process may utilize the energy director positioned at a 60-degree angle to control the melted area and create a step joint between the electrode pill 410 and the receiver 406. In some cases, the ultrasonic welding may create a 360-degree seal around the perimeter of the electrode pill 410, providing a mechanically robust attachment that protects internal electronics and enables reliable transmission of neuromuscular signals from the sensors to the capsule 120 of the wrist-wearable device 726 within the system.

(G8) In some embodiments of any of G1-G7, the second band section is configured to magnetically couple with the first band section in a plurality of locations along the first band section such that a circumference of the band of the wrist-wearable device is adjustable to a plurality of wrist sizes.

For example, as shown in FIG. 1, the continuous elongated strip configuration of the first attachment magnet extending along the first band section 100 may allow the magnetic-attachment structures 154a-154e of the second band section 150 to engage at multiple positions along the length of the first band section 100. The adjustable coupling capability may accommodate wrist circumferences ranging from small to large sizes by allowing a user to position any of the magnetic-attachment structures 154a-154e at different locations along the first attachment magnet. In some cases, as illustrated in the system shown in FIG. 7A, the adjustable circumference of the wrist-wearable device 726 may enable the device to be worn comfortably by the user 702 while maintaining consistent contact between the neuromuscular-signal sensors and the user's skin for accurate gesture detection during interactions with the AR device 728 and other components of the extended reality system.

(H1) In accordance with some embodiments, a wrist-wearable device includes a first band section of a band of the wrist-wearable device, a second band section of the band of the wrist-wearable device, at least one or more neuromuscular-signal sensors, and a flexible printed circuit board (FPCB), where the first band section is overmolded onto a first side of the FPCB of the wrist-wearable device, the second band section is overmolded onto a second side, opposite the first side, of the FPCB, and the at least one or more neuromuscular-signal sensors are coupled to the FPCB.

For example, as shown in FIG. 2, the wrist-wearable device may include the first band section 100 that is overmolded onto a first side of an internal electronics stack that includes a flexible printed circuit board. The manufacturing process may utilize a two-shot overmolding technique where the first shot portion 212 is overmolded onto the top portion of internal electronics stack 210a using a liquid injection molding process, followed by the second shot portion 214 being overmolded onto the bottom portion of internal electronics stack 210b. In some cases, as illustrated in FIG. 3, the flexible printed circuit assembly 310 may be positioned within the internal electronics stack such that the flexible printed circuit 306 becomes encapsulated within the first shot portion 212 and the second shot portion 214 during the overmolding process. The neuromuscular-signal sensors 106a-106f may be coupled to the FPCB through electrical connections that extend through the flexible printed circuit 306, enabling signal transmission from the sensors to processing components of the wrist-wearable device. As shown in FIG. 2, after the overmolding process, the internal electronics stack may be fully encapsulated inside the first shot portion 212 and the second shot portion 214, with only areas dedicated to assembly and integration of sensors and integration to the capsule 120 portion exposed after overmolding.

(H2) In some embodiments of H1, the one or more neuromuscular-signal sensors are coupled to the FPCB by at least one of ultrasonic welding, heat-activated film, or liquid-dispensed adhesive.

For example, as shown in FIG. 4, the neuromuscular-signal sensors 106a-106f may be coupled to the FPCB through ultrasonic welding, where the electrode pill 410 is attached to the receiver 406 by melting thermoplastic components together via high-frequency vibration. The ultrasonic welding process may utilize the energy director positioned at a 60-degree angle to control the melted area and create a step joint between the electrode pill 410 and the receiver 406. In some cases, heat-activated film may be used as an alternative coupling method, where a thermoplastic adhesive film is positioned between the neuromuscular-signal sensor and the FPCB and activated through the application of heat to create a bond. Liquid-dispensed adhesive may also be employed, where a liquid adhesive is precisely dispensed onto contact areas between the sensor and the FPCB and subsequently cured to form a mechanical and electrical connection. The selection of coupling method may depend on factors such as desired bond strength, manufacturing throughput requirements, and the specific materials used in the sensor and FPCB construction.

(H3) In some embodiments of any of H1-H2, the wrist-wearable device further includes a closure mechanism coupled to the first band section such that while the closure mechanism is in a locked position, the second band section is retained relative to the closure mechanism, and while the closure mechanism is in an unlocked position, the second band section can pass through the closure mechanism while tension is maintained on the second band section.

For example, as shown in FIG. 5, the wrist-wearable device 190 may include the band closure mechanism 502 coupled to the first band portion 500, where the band closure mechanism 502 is configured to receive the second band portion 550 and adjust between an unlocked state and a locked state. While the band closure mechanism 502 is in the locked state, the second band portion 550 may be retained in position relative to the closure mechanism and cannot slide through the band closure mechanism 502, thereby maintaining a fixed circumference for the wrist-wearable device 190. In some cases, while the band closure mechanism 502 is in the unlocked state, the second band portion 550 may slide through the band closure mechanism 502 with some resistance while tension is maintained on the second band portion 550, allowing a user to adjust the fit of the wrist-wearable device without completely removing the device from their wrist. The band closure mechanism 502 may provide tactile feedback during adjustment and may be configured to hold tension between the first band portion 500 and the second band portion 550 when transitioning between the unlocked and locked states.

(H4) In some embodiments of any of H1-H3, the first band section further includes a layer including a liquid crystal polymer (LCP) based fiber configured to provide stiffness for the first band section, and one or more receivers configured to couple to each respective neuromuscular-signal sensor.

For example, as shown in FIG. 3, the first band section may include the strain relief layer 302 comprising a manufactured fiber spun from a liquid crystal polymer, which may be configured to provide structural stiffness for the first band section while maintaining flexibility during bending. The strain relief layer 302 may reduce mechanical stress on the flexible printed circuit assembly 310 and the neuromuscular-signal sensors during flexing of the first band section, thereby prolonging the operational life of the wrist-wearable device. As illustrated in FIG. 4, the first band section 100 may further include one or more receivers 406 configured to couple to each respective neuromuscular-signal sensor from the sensors 106a-106f. In some cases, the receivers 406 may be injection molded and positioned within the first band section 100 to provide mounting locations for the neuromuscular-signal sensors, where each receiver 406 is configured to receive a corresponding sensor and facilitate both mechanical attachment and electrical connectivity between the sensor and the FPCB.

(H5) In some embodiments of any of H1-H4, each neuromuscular-signal sensor is configured to couple with each respective receiver via ultrasonic welding.

For example, as shown in FIG. 4, each neuromuscular-signal sensor from the sensors 106a-106f may be configured to couple with a respective receiver 406 via ultrasonic welding, which includes melting thermoplastic components of the electrode pill 410 and the receiver 406 together via high-frequency vibration. The ultrasonic welding process may utilize the energy director positioned at a 60-degree angle to control the melted area and create a step joint between the electrode pill 410 and the receiver 406. In some cases, the ultrasonic welding may create a 360-degree seal around the perimeter of the electrode pill 410, providing a mechanically robust attachment that protects internal electronics and enables reliable transmission of neuromuscular signals from the sensors to the capsule 120 of the wrist-wearable device. The sealed design may allow for continued band function during specific use cases such as hand washing, exposure to rain, exercising, or swimming, where the wrist-wearable device may be exposed to water or sweat.

(H6) In some embodiments of any of H1-H5, the second band section is configured to self-align when coupled with the first band section.

For example, as shown in FIG. 1, when the first band section 100 and the second band section 150 are brought together, the band sections may automatically adjust their relative positions to achieve proper alignment through magnetic forces. At the first point in time 192, the first band section 100 and the second band section 150 may be initially positioned in a misaligned configuration, where magnetic portions with the same polarity are adjacent to one another, creating repulsive forces between the band sections. In some cases, the repulsive forces may cause the band sections to shift relative to one another until opposing polarity portions align properly, as illustrated at the second point in time 194 where the band sections are properly aligned. The self-alignment mechanism may ensure that the neuromuscular-signal sensors 106a-106f maintain proper contact with the user's wrist without requiring manual adjustment by the user, thereby improving the ease of donning the wrist-wearable device and ensuring consistent sensor performance.

(I1) In accordance with some embodiments, a method of manufacturing a wrist-wearable device includes overmolding a first band section onto a first side of a flexible printed circuit board (FPCB) of the wrist-wearable device, overmolding a second band section onto a second side, opposite the first side, of the FPCB, and coupling at least one or more neuromuscular-signal sensors to the FPCB.

For example, as shown in FIG. 2, the manufacturing method may include overmolding the first shot portion 212 onto the top portion of internal electronics stack 210a that includes the FPCB using a liquid injection molding process, followed by overmolding the second shot portion 214 onto the bottom portion of internal electronics stack 210b. The overmolding process may fully encapsulate the FPCB within the first band section 100 and the second band section, with only the connection point 202 and areas dedicated to sensor assembly remaining exposed. In some cases, as illustrated in FIG. 3, the flexible printed circuit assembly 310 may be positioned within the internal electronics stack prior to overmolding such that the flexible printed circuit 306 becomes encapsulated during the overmolding process. The neuromuscular-signal sensors 106a-106f may be coupled to the FPCB after the overmolding process is complete, where the coupling may establish both mechanical attachment and electrical connectivity between the sensors and the FPCB to enable signal transmission to processing components of the wrist-wearable device.

(I2) In some embodiments of I1, the method further comprises coupling the at least one or more neuromuscular-signal sensors to the FPCB via at least one of ultrasonic welding, heat-activated film, or liquid-dispensed adhesive.

For example, as shown in FIG. 4, the coupling of the neuromuscular-signal sensors 106a-106f to the FPCB may be performed using ultrasonic welding, where the electrode pill 410 is attached to the receiver 406 by melting thermoplastic components together via high-frequency vibration. The ultrasonic welding process may utilize the energy director positioned at a 60-degree angle to control the melted area and create a step joint between the electrode pill 410 and the receiver 406, resulting in a 360-degree seal around the perimeter of the electrode pill 410. In some cases, heat-activated film may be used as an alternative coupling method, where a thermoplastic adhesive film is positioned between the neuromuscular-signal sensor and the FPCB and activated through the application of heat to create a bond. Liquid-dispensed adhesive may also be employed, where a liquid adhesive is precisely dispensed onto contact areas between the sensor and the FPCB and subsequently cured to form a mechanical and electrical connection. The selection of coupling method may depend on factors such as desired bond strength, manufacturing throughput requirements, and the specific materials used in the sensor and FPCB construction.

(I3) In some embodiments of any of I1-I2, the method further comprises overmolding the first band section and overmolding the second band section using a liquid injection molding process.

For example, as shown in FIG. 2, the overmolding of the first shot portion 212 onto the top portion of internal electronics stack 210a and the overmolding of the second shot portion 214 onto the bottom portion of internal electronics stack 210b may be performed using a liquid injection molding process. The liquid injection molding process may allow for precise control of material flow and thickness during the overmolding operation, enabling the creation of thin-walled structures that encapsulate the FPCB and other electronic components. In some cases, the liquid injection molding process may be performed sequentially, where the first shot portion 212 is molded and allowed to cure before the second shot portion 214 is molded onto the opposite side of the internal electronics stack. The liquid injection molding process may provide advantages such as improved material properties, reduced cycle times, and the ability to create complex geometries with consistent wall thicknesses throughout the first band section 100.

(I4) In some embodiments of any of I1-I3, the method further comprises forming the first band section and the second band section of liquid silicone rubber material.

For example, as shown in FIG. 2, the first shot portion 212 and the second shot portion 214 may be formed from liquid silicone rubber material that is injected during the liquid injection molding process. The liquid silicone rubber material may provide flexibility, improved skin feel, and chemical resistance for the first band section 100 and the second band section. In some cases, the liquid silicone rubber material may be selected to maintain a thin band structure while providing sufficient durability for daily wear of the wrist-wearable device. The liquid silicone rubber material may also be biocompatible and suitable for prolonged contact with the skin of a user, making it appropriate for use in a wearable device that includes neuromuscular-signal sensors 106a-106f that require consistent skin contact for accurate signal detection.

(I5) In some embodiments of any of I1-I4, after overmolding the first band section and the second band section, the method further comprises fully encapsulating the FPCB within the first band section and the second band section, with areas dedicated to assembly of the one or more neuromuscular-signal sensors and integration to a capsule portion being exposed.

For example, as shown in FIG. 2, after the overmolding process is complete, the internal electronics stack including the FPCB may be fully encapsulated inside the first shot portion 212 and the second shot portion 214, with only the connection point 202 and areas dedicated to assembly and integration of sensors remaining exposed. The full encapsulation of the FPCB may protect the electronic components from environmental factors such as moisture, dust, and mechanical stress during use of the wrist-wearable device. In some cases, the exposed areas may be precisely defined during the overmolding process through the use of tooling features that prevent liquid silicone rubber material from flowing into regions where subsequent assembly operations will occur. The connection point 202 may remain exposed to facilitate subsequent attachment to the capsule 120 portion of the wrist-wearable device, while sensor assembly areas may remain exposed to allow for the coupling of the neuromuscular-signal sensors 106a-106f to the receivers 406 positioned within the first band section 100.

(I6) In some embodiments of any of I1-I5, a thickness of the first band section or the second band section after overmolding is less than 4 mm.

For example, as shown in FIG. 2, after the manufacturing process is complete, the first band section 100 may have a thickness of less than 4 mm, which may include the thickness of the first shot portion 212 at less than 2 mm and the second shot portion 214 at less than 1 mm. The thin profile of the first band section 100 may be achieved through the use of liquid injection molding with liquid silicone rubber material, which may allow for precise control of wall thicknesses during the overmolding process. In some cases, the second shot portion 214 thickness may include a wall thickness of less than 1 mm and a texture depth of less than 0.5 mm. The thin band structure may provide improved comfort for the user while maintaining sufficient structural integrity to protect the encapsulated FPCB and support the neuromuscular-signal sensors 106a-106f positioned along the first side of first band section 108.

(I7) In some embodiments of any of I1-I6, the method further comprises permanently magnetizing a magnetic rubber material of the first band section or second band section using a uni-directional field.

For example, as shown in FIG. 1, the method may include permanently magnetizing the magnetic rubber material that forms the first magnetic portion 104a, the second magnetic portion 104b, and the third magnetic portion 104c of the first band section 100 using a uni-directional field. The uni-directional field may be applied after the magnetic rubber material has been compression molded into the specific shape of the first band section 100, ensuring that the magnetic force is stronger in the direction of tail retention. In some cases, the magnetic rubber material may comprise liquid silicone rubber mixed with magnetic particles in a predetermined ratio to create a homogenous blend, and the uni-directional field may align the magnetic domains within the magnetic particles to create the desired magnetic polarity pattern. The permanent magnetization process may establish the first magnetic polarity of the first magnetic portion 104a and the second magnetic portion 104b and the second magnetic polarity of the third magnetic portion 104c, enabling the magnetic coupling functionality between the first band section 100 and the second band section 150 as illustrated at the second point in time 194.

### Example Extended-Reality Systems

FIGS. 7A, 7B, 7C-1, and 7C-2 illustrate example XR systems that include AR and MR systems, in accordance with some embodiments. FIG. 7A shows a first XR system 700a and first example user interactions using a wrist-wearable device 726, a head-wearable device (e.g., AR device 728), and/or an HIPD 742. FIG. 7B shows a second XR system 700b and second example user interactions using a wrist-wearable device 726, AR device 728, and/or an HIPD 742. FIGS. 7C-1 and 7C-2 show a third MR system 700c and third example user interactions using a wrist-wearable device 726, a head-wearable device (e.g., an MR device such as a VR device), and/or an HIPD 742. As the skilled artisan will appreciate upon reading the descriptions provided herein, the above-example AR and MR systems (described in detail below) can perform various functions and/or operations.

The wrist-wearable device 726, the head-wearable devices, and/or the HIPD 742 can communicatively couple via a network 725 (e.g., cellular, near field, Wi-Fi, personal area network, wireless LAN). Additionally, the wrist-wearable device 726, the head-wearable device, and/or the HIPD 742 can also communicatively couple with one or more servers 730, computers 740 (e.g., laptops, computers), mobile devices 750 (e.g., smartphones, tablets), and/or other electronic devices via the network 725 (e.g., cellular, near field, Wi-Fi, personal area network, wireless LAN). Similarly, a smart textile-based garment, when used, can also communicatively couple with the wrist-wearable device 726, the head-wearable device(s), the HIPD 742, the one or more servers 730, the computers 740, the mobile devices 750, and/or other electronic devices via the network 725 to provide inputs.

Turning to FIG. 7A, a user 702 is shown wearing the wrist-wearable device 726 and the AR device 728 and having the HIPD 742 on their desk. The wrist-wearable device 726, the AR device 728, and the HIPD 742 facilitate user interaction with an AR environment. In particular, as shown by the first AR system 700a, the wrist-wearable device 726, the AR device 728, and/or the HIPD 742 cause presentation of one or more avatars 704, digital representations of contacts 706, and virtual objects 708. As discussed below, the user 702 can interact with the one or more avatars 704, digital representations of the contacts 706, and virtual objects 708 via the wrist-wearable device 726, the AR device 728, and/or the HIPD 742. In addition, the user 702 is also able to directly view physical objects in the environment, such as a physical table 729, through transparent lens(es) and waveguide(s) of the AR device 728. Alternatively, an MR device could be used in place of the AR device 728 and a similar user experience can take place, but the user would not be directly viewing physical objects in the environment, such as table 729, and would instead be presented with a virtual reconstruction of the table 729 produced from one or more sensors of the MR device (e.g., an outward-facing camera capable of recording the surrounding environment).

The user 702 can use any of the wrist-wearable device 726, the AR device 728 (e.g., through physical inputs at the AR device and/or built-in motion tracking of a user's extremities), a smart-textile garment, externally mounted extremity tracking device, the HIPD 742 to provide user inputs, etc. For example, the user 702 can perform one or more hand gestures that are detected by the wrist-wearable device 726 (e.g., using one or more EMG sensors and/or IMUs built into the wrist-wearable device) and/or AR device 728 (e.g., using one or more image sensors or cameras) to provide a user input. Alternatively, or additionally, the user 702 can provide a user input via one or more touch surfaces of the wrist-wearable device 726, the AR device 728, and/or the HIPD 742, and/or voice commands captured by a microphone of the wrist-wearable device 726, the AR device 728, and/or the HIPD 742. The wrist-wearable device 726, the AR device 728, and/or the HIPD 742 includes an artificially intelligent digital assistant to help the user in providing a user input (e.g., completing a sequence of operations, suggesting different operations or commands, providing reminders, confirming a command). For example, the digital assistant can be invoked through an input occurring at the AR device 728 (e.g., via an input at a temple arm of the AR device 728). In some embodiments, the user 702 can provide a user input via one or more facial gestures and/or facial expressions. For example, cameras of the wrist-wearable device 726, the AR device 728, and/or the HIPD 742 can track the user 702's eyes for navigating a user interface.

The wrist-wearable device 726, the AR device 728, and/or the HIPD 742 can operate alone or in conjunction to allow the user 702 to interact with the AR environment. In some embodiments, the HIPD 742 is configured to operate as a central hub or control center for the wrist-wearable device 726, the AR device 728, and/or another communicatively coupled device. For example, the user 702 can provide an input to interact with the AR environment at any of the wrist-wearable device 726, the AR device 728, and/or the HIPD 742, and the HIPD 742 can identify one or more back-end and front-end tasks to cause the performance of the requested interaction and distribute instructions to cause the performance of the one or more back-end and front-end tasks at the wrist-wearable device 726, the AR device 728, and/or the HIPD 742. In some embodiments, a back-end task is a background-processing task that is not perceptible by the user (e.g., rendering content, decompression, compression, application-specific operations), and a front-end task is a user-facing task that is perceptible to the user (e.g., presenting information to the user, providing feedback to the user). The HIPD 742 can perform the back-end tasks and provide the wrist-wearable device 726 and/or the AR device 728 operational data corresponding to the performed back-end tasks such that the wrist-wearable device 726 and/or the AR device 728 can perform the front-end tasks. In this way, the HIPD 742, which has more computational resources and greater thermal headroom than the wrist-wearable device 726 and/or the AR device 728, performs computationally intensive tasks and reduces the computer resource utilization and/or power usage of the wrist-wearable device 726 and/or the AR device 728.

In the example shown by the first AR system 700a, the HIPD 742 identifies one or more back-end tasks and front-end tasks associated with a user request to initiate an AR video call with one or more other users (represented by the avatar 704 and the digital representation of the contact 706) and distributes instructions to cause the performance of the one or more back-end tasks and front-end tasks. In particular, the HIPD 742 performs back-end tasks for processing and/or rendering image data (and other data) associated with the AR video call and provides operational data associated with the performed back-end tasks to the AR device 728 such that the AR device 728 performs front-end tasks for presenting the AR video call (e.g., presenting the avatar 704 and the digital representation of the contact 706).

In some embodiments, the HIPD 742 can operate as a focal or anchor point for causing the presentation of information. This allows the user 702 to be generally aware of where information is presented. For example, as shown in the first AR system 700a, the avatar 704 and the digital representation of the contact 706 are presented above the HIPD 742. In particular, the HIPD 742 and the AR device 728 operate in conjunction to determine a location for presenting the avatar 704 and the digital representation of the contact 706. In some embodiments, information can be presented within a predetermined distance from the HIPD 742 (e.g., within five meters). For example, as shown in the first AR system 700a, virtual object 708 is presented on the desk some distance from the HIPD 742. Similar to the above example, the HIPD 742 and the AR device 728 can operate in conjunction to determine a location for presenting the virtual object 708. Alternatively, in some embodiments, presentation of information is not bound by the HIPD 742. More specifically, the avatar 704, the digital representation of the contact 706, and the virtual object 708 do not have to be presented within a predetermined distance of the HIPD 742. While an AR device 728 is described working with an HIPD, an MR headset can be interacted with in the same way as the AR device 728.

User inputs provided at the wrist-wearable device 726, the AR device 728, and/or the HIPD 742 are coordinated such that the user can use any device to initiate, continue, and/or complete an operation. For example, the user 702 can provide a user input to the AR device 728 to cause the AR device 728 to present the virtual object 708 and, while the virtual object 708 is presented by the AR device 728, the user 702 can provide one or more hand gestures via the wrist-wearable device 726 to interact and/or manipulate the virtual object 708. While an AR device 728 is described working with a wrist-wearable device 726, an MR headset can be interacted with in the same way as the AR device 728.

### Integration of Artificial Intelligence With XR Systems

FIG. 7A illustrates an interaction in which an artificially intelligent virtual assistant can assist in requests made by a user 702. The Artificial Intelligence (AI) virtual assistant can be used to complete open-ended requests made through natural language inputs by a user 702. For example, in FIG. 7A the user 702 makes an audible request 744 to summarize the conversation and then share the summarized conversation with others in the meeting. In addition, the AI virtual assistant is configured to use sensors of the XR system (e.g., cameras of an XR headset, microphones, and various other sensors of any of the devices in the system) to provide contextual prompts to the user for initiating tasks.

FIG. 7A also illustrates an example neural network 752 used in Artificial Intelligence applications. Uses of AI are varied and encompass many different aspects of the devices and systems described herein. AI capabilities cover a diverse range of applications and deepen interactions between the user 702 and user devices (e.g., the AR device 728, an MR device 732, the HIPD 742, the wrist-wearable device 726). The AI discussed herein can be derived using many different training techniques. While the primary AI model example discussed herein is a neural network, other AI models can be used. Non-limiting examples of AI models include artificial neural networks (ANNs), deep neural networks (DNNs), convolution neural networks (CNNs), recurrent neural networks (RNNs), large language models (LLMs), long short-term memory networks, transformer models, decision trees, random forests, support vector machines, k-nearest neighbors, genetic algorithms, Markov models, Bayesian networks, fuzzy logic systems, and deep reinforcement learnings, etc. The AI models can be implemented at one or more of the user devices, and/or any other devices described herein. For devices and systems herein that employ multiple AI models, different models can be used, depending on the task. For example, for a natural-language AI virtual assistant, an LLM can be used, and for the object detection of a physical environment, a DNN can be used instead.

In another example, an AI virtual assistant can include many different AI models, and based on the user's request, multiple AI models may be employed (concurrently, sequentially or a combination thereof). For example, an LLM-based AI model can provide instructions for helping a user follow a recipe and the instructions can be based in part on another AI model that is derived from an ANN, a DNN, an RNN, etc. that is capable of discerning what part of the recipe the user is on (e.g., object and scene detection).

As AI training models evolve, the operations and experiences described herein could potentially be performed with different models other than those listed above, and a person skilled in the art would understand that the list above is non-limiting.

A user 702 can interact with an AI model through natural language inputs captured by a voice sensor, text inputs, or any other input modality that accepts natural language and/or a corresponding voice sensor module. In another instance, input is provided by tracking the eye gaze of a user 702 via a gaze tracker module. Additionally, the AI model can also receive inputs beyond those supplied by a user 702. For example, the AI can generate its response further based on environmental inputs (e.g., temperature data, image data, video data, ambient light data, audio data, GPS location data, inertial measurement (i.e., user motion) data, pattern recognition data, magnetometer data, depth data, pressure data, force data, neuromuscular data, heart rate data, temperature data, sleep data) captured in response to a user request by various types of sensors and/or their corresponding sensor modules. The sensors'data can be retrieved entirely from a single device (e.g., AR device 728) or from multiple devices that are in communication with each other (e.g., a system that includes at least two of an AR device 728, an MR device 732, the HIPD 742, the wrist-wearable device 726, etc.). The AI model can also access additional information (e.g., one or more servers 730, the computers 740, the mobile devices 750, and/or other electronic devices) via a network 725.

A non-limiting list of AI-enhanced functions includes but is not limited to image recognition, speech recognition (e.g., automatic speech recognition), text recognition (e.g., scene text recognition), pattern recognition, natural language processing and understanding, classification, regression, clustering, anomaly detection, sequence generation, content generation, and optimization. In some embodiments, AI-enhanced functions are fully or partially executed on cloud-computing platforms communicatively coupled to the user devices (e.g., the AR device 728, an MR device 732, the HIPD 742, the wrist-wearable device 726) via the one or more networks. The cloud-computing platforms provide scalable computing resources, distributed computing, managed AI services, interference acceleration, pre-trained models, APIs and/or other resources to support comprehensive computations required by the AI-enhanced function.

Example outputs stemming from the use of an AI model can include natural language responses, mathematical calculations, charts displaying information, audio, images, videos, texts, summaries of meetings, predictive operations based on environmental factors, classifications, pattern recognitions, recommendations, assessments, or other operations. In some embodiments, the generated outputs are stored on local memories of the user devices (e.g., the AR device 728, an MR device 732, the HIPD 742, the wrist-wearable device 726), storage options of the external devices (servers, computers, mobile devices, etc.), and/or storage options of the cloud-computing platforms.

The AI-based outputs can be presented across different modalities (e.g., audio-based, visual-based, haptic-based, and any combination thereof) and across different devices of the XR system described herein. Some visual-based outputs can include the displaying of information on XR augments of an XR headset, user interfaces displayed at a wrist-wearable device, laptop device, mobile device, etc. On devices with or without displays (e.g., HIPD 742), haptic feedback can provide information to the user 702. An AI model can also use the inputs described above to determine the appropriate modality and device(s) to present content to the user (e.g., a user walking on a busy road can be presented with an audio output instead of a visual output to avoid distracting the user 702).

### Example Augmented Reality Interaction

FIG. 7B shows the user 702 wearing the wrist-wearable device 726 and the AR device 728 and holding the HIPD 742. In the second AR system 700b, the wrist-wearable device 726, the AR device 728, and/or the HIPD 742 are used to receive and/or provide one or more messages to a contact of the user 702. In particular, the wrist-wearable device 726, the AR device 728, and/or the HIPD 742 detect and coordinate one or more user inputs to initiate a messaging application and prepare a response to a received message via the messaging application.

In some embodiments, the user 702 initiates, via a user input, an application on the wrist-wearable device 726, the AR device 728, and/or the HIPD 742 that causes the application to initiate on at least one device. For example, in the second AR system 700b, the user 702 performs a hand gesture associated with a command for initiating a messaging application (represented by messaging user interface 712); the wrist-wearable device 726 detects the hand gesture; and, based on a determination that the user 702 is wearing the AR device 728, causes the AR device 728 to present a messaging user interface 712 of the messaging application. The AR device 728 can present the messaging user interface 712 to the user 702 via its display (e.g., as shown by user 702's field of view 710). In some embodiments, the application is initiated and can be run on the device (e.g., the wrist-wearable device 726, the AR device 728, and/or the HIPD 742) that detects the user input to initiate the application, and the device provides another device operational data to cause the presentation of the messaging application. For example, the wrist-wearable device 726 can detect the user input to initiate a messaging application, initiate and run the messaging application, and provide operational data to the AR device 728 and/or the HIPD 742 to cause presentation of the messaging application. Alternatively, the application can be initiated and run at a device other than the device that detected the user input. For example, the wrist-wearable device 726 can detect the hand gesture associated with initiating the messaging application and cause the HIPD 742 to run the messaging application and coordinate the presentation of the messaging application.

Further, the user 702 can provide a user input provided at the wrist-wearable device 726, the AR device 728, and/or the HIPD 742 to continue and/or complete an operation initiated at another device. For example, after initiating the messaging application via the wrist-wearable device 726 and while the AR device 728 presents the messaging user interface 712, the user 702 can provide an input at the HIPD 742 to prepare a response (e.g., shown by the swipe gesture performed on the HIPD 742). The user 702's gestures performed on the HIPD 742 can be provided and/or displayed on another device. For example, the user 702's swipe gestures performed on the HIPD 742 are displayed on a virtual keyboard of the messaging user interface 712 displayed by the AR device 728.

In some embodiments, the wrist-wearable device 726, the AR device 728, the HIPD 742, and/or other communicatively coupled devices can present one or more notifications to the user 702. The notification can be an indication of a new message, an incoming call, an application update, a status update, etc. The user 702 can select the notification via the wrist-wearable device 726, the AR device 728, or the HIPD 742 and cause presentation of an application or operation associated with the notification on at least one device. For example, the user 702 can receive a notification that a message was received at the wrist-wearable device 726, the AR device 728, the HIPD 742, and/or other communicatively coupled device and provide a user input at the wrist-wearable device 726, the AR device 728, and/or the HIPD 742 to review the notification, and the device detecting the user input can cause an application associated with the notification to be initiated and/or presented at the wrist-wearable device 726, the AR device 728, and/or the HIPD 742.

While the above example describes coordinated inputs used to interact with a messaging application, the skilled artisan will appreciate upon reading the descriptions that user inputs can be coordinated to interact with any number of applications, including, but not limited to, gaming applications, social media applications, camera applications, web-based applications, financial applications, etc. For example, the AR device 728 can present to the user 702 game application data, and the HIPD 742 can use a controller to provide inputs to the game. Similarly, the user 702 can use the wrist-wearable device 726 to initiate a camera of the AR device 728, and the user can use the wrist-wearable device 726, the AR device 728, and/or the HIPD 742 to manipulate the image capture (e.g., zoom in or out, apply filters) and capture image data.

While an AR device 728 is shown being capable of certain functions, it is understood that an AR device can be an AR device with varying functionalities based on costs and market demands. For example, an AR device may include a single output modality such as an audio output modality. In another example, the AR device may include a low-fidelity display as one of the output modalities, where simple information (e.g., text and/or low-fidelity images/video) is capable of being presented to the user. In yet another example, the AR device can be configured with face-facing light emitting diodes (LEDs) configured to provide a user with information, e.g., an LED around the right-side lens can illuminate to notify the wearer to turn right while directions are being provided, or an LED on the left-side lens can illuminate to notify the wearer to turn left while directions are being provided. In another embodiment, the AR device can include an outward-facing projector such that information (e.g., text information, media) may be displayed on the palm of a user's hand or other suitable surface (e.g., a table, whiteboard). In yet another embodiment, information may also be provided by locally dimming portions of a lens to emphasize portions of the environment in which the user's attention should be directed. Some AR devices can present AR augments either monocularly or binocularly (e.g., an AR augment can be presented at only a single display associated with a single lens as opposed to presenting an AR augmented at both lenses to produce a binocular image). In some instances, an AR device capable of presenting AR augments binocularly can optionally display AR augments monocularly as well (e.g., for power-saving purposes or other presentation considerations). These examples are non-exhaustive, and features of one AR device described above can be combined with features of another AR device described above. While features and experiences of an AR device have been described generally in the preceding sections, it is understood that the described functionalities and experiences can be applied in a similar manner to an MR headset, which is described below in the proceeding sections.

### Example Mixed Reality Interaction

Turning to FIGS. 7C-1 and 7C-2, the user 702 is shown wearing the wrist-wearable device 726 and an MR device 732 (e.g., a device capable of providing either an entirely VR experience or an MR experience that displays object(s) from a physical environment at a display of the device) and holding the HIPD 742. In the third AR system 700c, the wrist-wearable device 726, the MR device 732, and/or the HIPD 742 are used to interact within an MR environment, such as a VR game or other MR/VR application. While the MR device 732 presents a representation of a VR game (e.g., first MR game environment 720) to the user 702, the wrist-wearable device 726, the MR device 732, and/or the HIPD 742 detect and coordinate one or more user inputs to allow the user 702 to interact with the VR game.

In some embodiments, the user 702 can provide a user input via the wrist-wearable device 726, the MR device 732, and/or the HIPD 742 that causes an action in a corresponding MR environment. For example, the user 702 in the third MR system 700c (shown in FIG. 7C-1) raises the HIPD 742 to prepare for a swing in the first MR game environment 720. The MR device 732, responsive to the user 702 raising the HIPD 742, causes the MR representation of the user 722 to perform a similar action (e.g., raise a virtual object, such as a virtual sword 724). In some embodiments, each device uses respective sensor data and/or image data to detect the user input and provide an accurate representation of the user 702's motion. For example, image sensors (e.g., SLAM cameras or other cameras) of the HIPD 742 can be used to detect a position of the HIPD 742 relative to the user 702's body such that the virtual object can be positioned appropriately within the first MR game environment 720; sensor data from the wrist-wearable device 726 can be used to detect a velocity at which the user 702 raises the HIPD 742 such that the MR representation of the user 722 and the virtual sword 724 are synchronized with the user 702's movements; and image sensors of the MR device 732 can be used to represent the user 702's body, boundary conditions, or real-world objects within the first MR game environment 720.

In FIG. 7C-2, the user 702 performs a downward swing while holding the HIPD 742. The user 702's downward swing is detected by the wrist-wearable device 726, the MR device 732, and/or the HIPD 742, and a corresponding action is performed in the first MR game environment 720. In some embodiments, the data captured by each device is used to improve the user's experience within the MR environment. For example, sensor data of the wrist-wearable device 726 can be used to determine a speed and/or force at which the downward swing is performed and image sensors of the HIPD 742 and/or the MR device 732 can be used to determine a location of the swing and how it should be represented in the first MR game environment 720, which, in turn, can be used as inputs for the MR environment (e.g., game mechanics, which can use detected speed, force, locations, and/or aspects of the user 702's actions to classify a user's inputs (e.g., user performs a light strike, hard strike, critical strike, glancing strike, miss) or calculate an output (e.g., amount of damage)).

FIG. 7C-2 further illustrates that a portion of the physical environment is reconstructed and displayed at a display of the MR device 732 while the MR game environment 720 is being displayed. In this instance, a reconstruction of the physical environment 746 is displayed in place of a portion of the MR game environment 720 when object(s) in the physical environment are potentially in the path of the user (e.g., a collision with the user and an object in the physical environment are likely). Thus, this example MR game environment 720 includes (i) an immersive VR portion 748 (e.g., an environment that does not have a corollary counterpart in a nearby physical environment) and (ii) a reconstruction of the physical environment 746 (e.g., table 729 and cup 752). While the example shown here is an MR environment that shows a reconstruction of the physical environment to avoid collisions, other uses of reconstructions of the physical environment can be used, such as defining features of the virtual environment based on the surrounding physical environment (e.g., a virtual column can be placed based on an object in the surrounding physical environment (e.g., a tree)).

While the wrist-wearable device 726, the MR device 732, and/or the HIPD 742 are described as detecting user inputs, in some embodiments, user inputs are detected at a single device (with the single device being responsible for distributing signals to the other devices for performing the user input). For example, the HIPD 742 can operate an application for generating the first MR game environment 720 and provide the MR device 732 with corresponding data for causing the presentation of the first MR game environment 720, as well as detect the user 702's movements (while holding the HIPD 742) to cause the performance of corresponding actions within the first MR game environment 720. Additionally or alternatively, in some embodiments, operational data (e.g., sensor data, image data, application data, device data, and/or other data) of one or more devices is provided to a single device (e.g., the HIPD 742) to process the operational data and cause respective devices to perform an action associated with processed operational data.

In some embodiments, the user 702 can wear a wrist-wearable device 726, an MR device 732, smart textile-based garments 738 (e.g., wearable haptic gloves), and/or hold an HIPD 742 device. In this embodiment, the wrist-wearable device 726, the MR device 732, and/or the smart textile-based garments 738 are used to interact within an MR environment (e.g., any AR or MR system described above in reference to FIGS. 7A-7B). While the MR device 732 presents a representation of an MR game (e.g., second MR game environment 720) to the user 702, the wrist-wearable device 726, the MR device 732, and/or the smart textile-based garments 738 detect and coordinate one or more user inputs to allow the user 702 to interact with the MR environment.

In some embodiments, the user 702 can provide a user input via the wrist-wearable device 726, an HIPD 742, the MR device 732, and/or the smart textile-based garments 738 that cause an action in a corresponding MR environment. In some embodiments, each device uses respective sensor data and/or image data to detect the user input and provide an accurate representation of the user 702's motion. While four different input devices are shown (e.g., a wrist-wearable device 726, an MR device 732, an HIPD 742, and a smart textile-based garment 738), each one of these input devices entirely on its own can provide inputs for fully interacting with the MR environment. For example, the wrist-wearable device can provide sufficient inputs on its own for interacting with the MR environment. In some embodiments, if multiple input devices are used (e.g., a wrist-wearable device and the smart textile-based garment 738), sensor fusion can be utilized to ensure inputs are correct. While multiple input devices are described, it is understood that other input devices can be used in conjunction or on their own instead, such as but not limited to external motion-tracking cameras, other wearable devices fitted to different parts of a user, apparatuses that allow for a user to experience walking in an MR environment while remaining substantially stationary in the physical environment, etc.

As described above, the data captured by each device is used to improve the user's experience within the MR environment. Although not shown, the smart textile-based garments 738 can be used in conjunction with an MR device and/or an HIPD 742.

While some experiences are described as occurring on an AR device and other experiences are described as occurring on an MR device, one skilled in the art would appreciate that experiences can be ported over from an MR device to an AR device, and vice versa.

Some definitions of devices and components that can be included in some or all of the example devices discussed are defined here for ease of reference. A skilled artisan will appreciate that certain types of the components described may be more suitable for a particular set of devices, and less suitable for a different set of devices. But subsequent reference to the components defined here should be considered to be encompassed by the definitions provided.

In some embodiments, example devices and systems, including electronic devices and systems, will be discussed. Such example devices and systems are not intended to be limiting, and one of skill in the art will understand that alternative devices and systems to the example devices and systems described herein may be used to perform the operations and construct the systems and devices that are described herein.

As described herein, an electronic device is a device that uses electrical energy to perform a specific function. It can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices and/or a subset of components of one or more electronic devices and facilitates communication and/or data processing and/or data transfer between the respective electronic devices and/or electronic components.

The foregoing descriptions of FIGS. 7A-7C-2 provided above are intended to augment the description provided in reference to FIGS. 1-6. While terms in the following description may not be identical to terms used in the foregoing description, a person having ordinary skill in the art would understand these terms to have the same meaning.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt in or opt out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

1. A wrist-wearable device including a band for sensing neuromuscular signals, the wrist-wearable device comprising:
a first band section;
a second band section;
a plurality of neuromuscular-signal sensors disposed within the first band section, wherein the neuromuscular-signal sensors are configured to be in contact with a wrist of a user on a first side of the first band section;
a first attachment magnet disposed within the first band section wherein the first attachment magnet having a first central magnetic portion with a first magnetic polarity disposed between a plurality of first lateral magnetic portions each having a second magnetic polarity; and
a second attachment magnet disposed within the second band section, wherein:
the second attachment magnet has a second central magnetic portion with the second magnetic polarity disposed between a plurality of second lateral magnetic portions each having the first magnetic polarity, wherein:
a magnetic attractive force between the first central magnetic portion of the first attachment magnet and respective second central magnetic portions of the second attachment magnet are configured to magnetically couple the second band section with the first band section.

2. The wrist-wearable device of claim 1, wherein:
the second band section is configured to magnetically repel the first band section while the band is in a first position, the first position including:
the first central magnetic portion of the first band section and the plurality of second lateral magnetic portions of the second attachment magnet are positioned such that there is a magnetic repulsive force therebetween to cause the band to adjust to a self-aligned position, or
the second central magnetic portion of each respective second attachment magnet and respective first lateral magnetic portions of the first attachment magnet are positioned such that there is a magnetic repulsive force therebetween to cause the band to adjust to the self-aligned position.

3. The wrist-wearable device of claim 1, wherein:
the plurality of second lateral magnetic portions of each respective second attachment magnet are spaced apart from each other.

4. The wrist-wearable device of claim 1, wherein the first attachment magnet is a continuous elongated strip along a length of the first band section.

5. The wrist-wearable device of claim 1, wherein the first attachment magnet comprises a magnetic rubber material formed from liquid silicone rubber mixed with magnetic particles.

6. The wrist-wearable device of claim 1, wherein the second attachment magnet are coupled by a connection layer comprising a strain relief layer formed of a manufactured fiber spun from a liquid crystal polymer.

7. The wrist-wearable device of claim 1, wherein the second band section further includes an end portion having magnetic properties configured to couple to the first band section.

8. The wrist-wearable device of claim 7, wherein the end portion of the second band section includes stronger magnetic properties than other portions of the second band section such that additional force is required to remove the end portion from the first band section.

9. (canceled)

10. (canceled)

11. The wrist-wearable device of claim 1, wherein the second band section is configured to magnetically couple with the first band section in a plurality of locations along the first band section such that a circumference of the band is adjustable to a plurality of wrist sizes.

12. The wrist-wearable device of claim 1, wherein the second band section is configured to self-align when coupled with the first band section.

13. The wrist-wearable device of claim 1, wherein the second band section does not include neuromuscular-signal sensors.

14. A wrist-wearable system comprising:
a capsule; and
a band comprising:
a first band section configured to couple to the capsule;
a second band section configured to couple to the capsule;
a plurality of neuromuscular-signal sensors disposed within the first band section, wherein the neuromuscular-signal sensors are configured to be in contact with a wrist of a user on a first side of the first band section;
a first attachment magnet disposed within the first band section wherein the first attachment magnet having a first magnetic portion with a first magnetic polarity and a second magnetic portion having a second magnetic polarity; and
a plurality of second attachment magnets disposed within the second band section, wherein each respective second attachment magnet having a third magnetic portion with the second magnetic polarity and lateralfourth magnetic eachportion having the first magnetic polarity, wherein:
a magnetic attractive force between the first central magnetic portion of the first attachment magnet and respective third magnetic portions of the plurality of second attachment magnets and the magnetic attractive force between the second magnetic portion of the first attachment magnet and respective plurality of fourth magnetic portions of the plurality of second attachment magnets are configured to magnetically couple to the second band section with the first band section.

15. The wrist-wearable system of claim 14, wherein:
the fourth magnetic portion of each respective second attachment magnet are a predetermined distance away from each other.

16. The wrist-wearable system of claim 14, wherein the first attachment magnet is continuous along a length of the first band section.

17. The wrist-wearable system of claim 14, wherein the capsule includes a capsule display and the capsule is configured to:
process one or more detected neuromuscular signals and based on the one or more detected neuromuscular signals:
determine one or more gestures performed by the user; and
display one or more actions associated with the one or more gestures.

18. The wrist-wearable system of claim 14, wherein the first band section further includes:
a layer including a manufactured fiber that is spun from an LCP configured to provide stiffness for the first band section; and
one or more receivers configured to couple to each respective neuromuscular-signal sensor.

19. The wrist-wearable system of claim 14, wherein each neuromuscular-signal sensor is configured to couple with each respective receiver via ultrasonic welding.

20. The wrist-wearable system of claim 14, wherein the second band section is configured to magnetically coupled with the first band section in a plurality of locations along the first band section such that a circumference of the band is adjustable to a plurality of wrist sizes.

21. The wrist-wearable system of claim 14, wherein the first band section is connected to a first side of the capsule via a first connection point, and the second band section is connected to a second side of the capsule, opposite the first side of the capsule, via a second connection point.

22. The wrist-wearable system of claim 14, wherein the capsule further includes a display configured to:
process one or more detected neuromuscular signals and based on the one or more detected neuromuscular signals:
determine one or more gestures performed by the user; and
display one or more actions associated with the one or more gestures on the display.
